SEQUENCE LISTING (4 sequences)

SEQ ID NO: 1 - DNA, 1080 bp, Gallus gallus
  CDS (0)...(1080)
atgcgttccgtcaggaagaggtggactgtgtgcaccataagtctcctcctcatcttctacaagactaaggagatcgcgcgcactgaggagcgccaggaggctccgctcgccggagatggtgagctgagtttgagtcgatcaatgatcaatagctctgataaaataatccgaaagggcggctccgctatcttccagcattctgtagaaggttggaaaatcaattctactttggtactggagataagaaagagtattctccgattcttggatgcggaaagagatgtctcagtggtcaagagcagctttaagccaggagatgtaatccattatgtactagacagacgtcgcaccctaaatatttctcaggatttgcacagccttctccctgaggtttccccaatgaagaatcgccgatttaaaacctgtgctgtagttggaaattctggcatccttctggacagcgggtgtggaaaagagattgatacccatgattttgttataaggtgcaatctagctcccgtggtggagtttgctgcagatgtgggaaataaatctgattttattaccatgaacccatcagttgtacaaagagcatttggaggctttcggaatgagagtgacagagaaaaatttgggcatagactatccatgctgaatgacagtgtcctttggatccctgctttcatggtcaaaggcggagagaagcatttggagtgggttaatgcattaatccttaagaataaattgaaagtgcgaaccgcctatccatcactgagacttattcatgctgtcagaggttactggctcacaaacaaggtccacatcaaacgacccagcactggtctcctcatgtacacgcttgccaccagattctgtgatgaaattcacctgtatggattttggccattcccaaaggatttacatggaaaaccagtcaaatatcattattacgatgatctgaagtatcggtacttttctaatgccagccctcacaggatgccattagagtttaaaacattatatgtattacataacagaggagcacttaagttaacaacgggcaagtgcgtgaagcaataa

SEQ ID NO: 2 - protein, 359 aa, Gallus gallus
MRSVRKRWTVCTISLLLIFYKTKEIARTEERQEAPLAGDGELSLSRSMINSSDKIIRKGGSAIFQHSVEGWKINSTLVLEIRKSILRFLDAERDVSVVKSSFKPGDVIHYVLDRRRTLNISQDLHSLLPEVSPMKNRRFKTCAVVGNSGILLDSGCGKEIDTHDFVIRCNLAPVVEFAADVGNKSDFITMNPSVVQRAFGGFRNESDREKFGHRLSMLNDSVLWIPAFMVKGGEKHLEWVNALILKNKLKVRTAYPSLRLIHAVRGYWLTNKVHIKRPSTGLLMYTLATRFCDEIHLYGFWPFPKDLHGKPVKYHYYDDLKYRYFSNASPHRMPLEFKTLYVLHNRGALKLTTGKCVKQ

SEQ ID NO: 3 - DNA, 1894 bp, Homo sapiens
  CDS (213)...(1292)
cgcaaacagggcgagaggtcgctgggcagcgttcgaggaccagagggagctcggccacagaagaccccagtgatctgatcccgggatcccggctccaagctctcctcgcattttacagatttcacccccgcgactatctccccaaaacggagcctttatatcaagagaaggtgcgggagctggggcaaccaggactttctcgggcacccaagatgcgctccattaggaagaggtggacgatctgcacaataagtctgctcctgatcttttataagacaaaagaaatagcaagaactgaggagcaccaggagacgcaactcatcggagatggtgaattgtctttgagtcggtcacttgtcaatagctctgataaaatcattcgaaaggctggctcttcaatcttccagcacaatgtagaaggttggaaaatcaattcctctttggtcctagagataaggaagaacatacttcgtttcttagatgcagaacgagatgtgtcagtggtcaagagcagttttaagcctggtgatgtcatacactatgtgcttgacaggcgccggacactaaacatttctcatgatctacatagcctcctacctgaagtttcaccaatgaagaatcgcaggtttaagacctgtgcagttgttggaaattctggcattctgttagacagtgaatgtggaaaggagattgacagtcacaattttgtaataaggtgtaatctagctcctgtggtggagtttgctgcagatgtgggaactaaatcagattttattaccatgaatccatcagttgtacaaagagcatttggaggctttcgaaatgagagtgacagagaaaaatttgtgcatagactttccatgctgaatgacagtgtcctttggattcctgctttcatggtcaaaggaggagagaagcacgtggagtgggttaatgcattaatccttaagaataaactgaaagtgcgaactgcctatccgtcattgagacttattcatgctgtcagaggttactggctgaccaacaaagttcctatcaaaagacccagcacaggtcttctcatgtatacacttgccacaagattctgtgatgaaattcacctgtatggattctggcccttccctaaggatttaaatggaaaagcggtcaaatatcattattatgatgacttaaaatataggtacttttccaatgcaagccctcacagaatgccattagaattcaaaacattaaatgtgctacataatagaggagctctaaaactgacaacaggaaagtgtgtaaagcaataaagcacattttgaaacaaacaatatgcacttcttttctgagatgcttccgaagatttgaaaataggatccaaaacacggctgggtttcagcatccaccaatgaactgaaaggtgaataaaggacgttcatgagaaatcgactaccagctgatgaaatacctgcaaagtgctctaaaaattaaatattttgactttaagggtcctagtaagtgccacttccactaagaatacagtttgaatgtataatcagtagtgtttacaagatccaacagtgcactcatcattagttaacaaagcaaatatgttcatcactgtcaggctgcccacagcaacaccaagcatattagaagaggaaccccaggaacgcaactcagaccttgggaaattaaaccatccttgtcagcagaagccaagatggaagcagtttgagcaatgaaatccgtaagattaaacaactcaagtaaatgcttcagtcaggactctgagtctgatcatgaattttatgttttaatttatgtttttttttttgtcttctggaatctcttttggtttggatattgggatgcttagaaatcctttctgagatgcatatgagtgaggaaa

SEQ ID NO: 4 - protein, 359 aa, Homo sapiens
MRSIRKRWTICTISLLLIFYKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIRCNLAPVVEFAADVGTKSDFITMNPSVVQRAFGGFRNESDREKFVHRLSMLNDSVLWIPAFMVKGGEKHVEWVNALILKNKLKVRTAYPSLRLIHAVRGYWLTNKVPIKRPSTGLLMYTLATRFCDEIHLYGFWPFPKDLNGKAVKYHYYDDLKYRYFSNASPHRMPLEFKTLNVLHNRGALKLTTGKCVKQ

(19) United States
(12) Patent Application Publication
(10) Pub. No.: US 2008/0095747 A1
(43) Pub. Date: Apr. 24, 2008

(54) Promotion of Cell Migration and Axon Regeneration in the Cns

(75) Inventors: Urs Rutishauser, New Kingston, NY (US); Athanasios K. Petridis, Kiel (DE); Abderrahman El Maarouf, New York, NY (US)

Correspondence Address: PROTEUS PATENT PRACTICE LLC, P.O. BOX 1867, NEW HAVEN, CT, 06508, US

(73) Assignee: Memorial Sloan Kettering Cancer Center, New York, NY (US)

(21) Appl. No.: 11/576,614

(22) PCT Filed: Oct. 5, 2005
(86) PCT No.: PCT/US05/36129

§ 371 Date: Apr. 4, 2007

Related U.S. Application Data
(60) Provisional application No. 60/616,339, filed on Oct. 5, 2004.
(60) Provisional application No. 60/616,422, filed on Oct. 6, 2004.
(60) Provisional application No. 60/706,705, filed on Aug. 9, 2005.

(51) Int. Cl. A61K 39/00; A61P 43/00; C07K 16/00; C12N 15/00

(52) U.S. Cl. 424/93.7; 424/94.5; 435/320.1; 435/375; 530/387.1

(57) ABSTRACT

The present invention relates broadly to methods for stimulating neural progenitor cell migration to certain regions of the nervous system where the neural progenitor cell naturally would not migrate. These methods have wide interest in fields related to the development of therapeutic approaches for addressing a broad range of neurodegenerative and demyelinating pathologies of the central nervous system. In order to accomplish these various outcomes, the invention provides modalities for introducing into a cell of the CNS a substance that promotes polysialylation of a protein component of the cell. This process underlies various methods of the invention, such as a method of polysialylating a protein of the cell, a method of promoting migration of a neural progenitor cell from a first region of a brain to a second region of the brain, or a method of promoting a neural progenitor cell originating in a first region of a brain to differentiate in a second region of the brain. The methods described above provide the basis for various therapeutic methods disclosed in the invention. In one example a method is disclosed of inhibiting the development of, treating, or ameliorating a neurological pathology in a subject, wherein the method includes introducing into brain cells, located in a path starting in a location close to where neural progenitor cells are located and ending in an area of the CNS where it is desirable that the neural progenitor cells migrate to, of the subject a substance that promotes polysialyltransferase activity that polysialylates a protein of the cells. In a second example a method is disclosed of inhibiting the development of, treating, or ameliorating a neurological pathology in a subject, including administering to the subject a substance that promotes polysialyltransferase activity in an amount effective to treat the pathology.

### FIELD OF THE INVENTION

The present invention relates generally to methods and compositions that provide enhanced migration of neural cells, or their extensions, from an originating region to a second region in which, or at which, a need for such cells and/or their extensions has been identified.

### BACKGROUND OF THE INVENTION

Central nervous system (CNS) progenitor cells are located in discrete regions of the adult mammalian CNS including the subventricular zone (SVZ) of the lateral ventricle and the dentate gyrus in the hippocampus. New neural progenitors are continuously born in the adult mammalian brain, where they migrate to replenish particular cell populations. Adult mammalian CNS progenitor cells, however, manifest only a limited ability to repair damage, such as may arise from various pathologies or traumas.

At early stages of CNS cell fate determination, progenitors express the polysialylated form of the neural cell adhesion molecule (PSA-NCAM). PSA is a long linear homopolymer of α-2,8-linked sialic acid that glycosylates the extracellular domain of NCAM. Polysialyltransferases (PSTs) including ST8SiaII/STX and ST8SiaIV/PST are enzymes that catalyze the addition of PSA to NCAM. The large hydrated volume of PSA is believed to produce steric encumbrance between apposing membranes to attenuate cell-cell interactions mediated by NCAM other surface receptors. In adult vertebrate brain, PSA occurs in discrete locations such as the hypothalamo-neurohypophyseal system, dentate gyrus and SVZ.

Although being preferentially committed to a restricted fate as either glial or neuronal cells, cultured PSA-NCAM(+) SVZ progenitors preserve a degree of multipotentiality. Precursors expressing PSA-NCAM can populate regions of the adult neocortex following injury in rodents. Polysialylation of NCAM by transfection of a PST in vivo induces remodeling of a PSA-negative tissue.

There are numerous CNS pathologies or diseases for which treatments are limited, and that offer poor prognosis for improvement and recovery. These include demyelinating diseases such as multiple sclerosis, leukoencephalopathies and myelinolysis, in which, in general, oligodendrocytes are depleted.

CNS axons fail to regrow and reinnervate their targets following lesion. However, central axons are intrinsically able to regenerate. It has been known for more than 100 years that initial sprouting of severed CNS axons does not progress afterwards. The CNS environment interferes with axon regeneration, and the glial scar that forms at the lesion site provides a molecular barrier to regeneration. This scar contains inhibitory molecules of the extracellular matrix that are produced by reactive astrocytes and oligodendrocyte precursors at and adjacent to the lesion site.

Brain lesions can arise in the cortex from both ischemic and hemorrhagic stroke, and from traumatic injury to the brain. Scarring and/or cell death in the regions of the lesion responds poorly to therapies intended to restore CNS tissues.

Traumatic injury to the CNS can lead to interruption of axon pathways, such as the corticospinal tract, formation of scar tissue at the site of the injury, and consequent loss of transmission of nerve impulses to distal synapses. For example, paralysis and impairment of voluntary muscle movement, as well as correlated defects, arise as a result of spinal cord injuries. At present there is no effective therapy to improve the prognosis for patients suffering such traumas.

From the summary presented above it is evident that there remains a strong need for methods of promoting migration of a progenitor cell from a zone of origin toward a distal zone, where it might differentiate and carry out a particular function. There is additionally a pressing need for methods to promote regrowth of axons from proximal loci to distal areas across the site of traumatic rupture of CNS axon pathways, such as the within the cortex or in the corticospinal tract. Furthermore, there is a need for effective therapies that promote regeneration of CNS tissue at the site of a lesion such as might arise from stroke or trauma. There further remains a need for regenerating axons to reestablish synapses with their targets distal to the lesion-induced scar. There further is a need for effective therapies for neurodegenerative and demyelinating diseases. The present invention addresses these and related needs.

### SUMMARY OF THE INVENTION

The present invention relates broadly to ministrations to certain portions of the central nervous system that provide enhanced migration of neural cells, or their extensions, from an originating region to a second region in which, or at which, a need for such cells and/or their extensions has been identified.

In a first aspect the invention provides a method of promoting migration of a first neural cell or portion thereof from an originating region toward a second region, wherein the method includes the step of contacting a second neural cell with a composition that is effective to decorate the second neural cell with a hydrated polymer. This second neural cell is located substantially within a zone of interest. Many significant embodiments of this method are described in the following paragraphs; other embodiments of comparable significance are also comprehended within the scope of the invention. In various embodiments, the zone of interest includes, in a first instance, the second region; or in a second instance, the originating region; or in a third instance, a region of space that includes at least the originating region, the second region and a volume extending between them. The originating region includes any region that is a source of neural progenitor cells, or a source of outgrowth of neural cell projections such as axons or dendrites. The second region includes any region that is an intended destination for the migration of progenitor or the extension of the neural cell projection.

In additional embodiments of the method the second neural cell that becomes decorated with the hydrated polymer may be a glial cell, or it may be a neuronal cell.

In still further embodiments the hydrated polymer may include a hydrogel. Additionally the hydrated polymer may include a polysaccharide or any derivative of a polysaccharide; examples of polysaccharides include a polysialic acid, a hyaluronic acid, a polylactosamine, or a derivative of any of them.

In still additional embodiments of the method the composition effective to decorate the second cell includes a polynucleotide encoding a polypeptide having activity that synthesizes a hydrated polymer bound to an external surface of the second neural cell, or a complement thereof. This polynucleotide may, in further embodiments, be incorporated into a plasmid, vector, or virus particle that enters the second neural cell and expresses the polynucleotide therein. In still additional embodiments the encoded polypeptide includes a polysialyltransferase, an ortholog thereof, a variant thereof, or a fragment of any of them. The activity of the polypeptide is such that it synthesizes a hydrated polymer bound to a moiety on the external surface of the second neural cell. In yet further embodiments the moiety to which the hydrated polymer binds is a protein, such as a neural cell adhesion molecule.

In yet further embodiments of the method the composition effective to decorate the second cell includes a polysialyltransferase, or any substance that is an ortholog thereof, a variant thereof, a fragment of any of the foregoing, a peptidomimetic, or a mimetic compound, that exhibits polysialyltransferase activity. The activity is such that it synthesizes a hydrated polymer bound to a moiety on the external surface of the second neural cell. In yet further embodiments the moiety to which the hydrated polymer binds is a protein, such as a neural cell adhesion molecule.

In additional embodiments of the method, the composition includes a conjugate including the hydrated polymer bound to a targeting means wherein the targeting means binds to a moiety of the external surface of the second neural cell. In still additional embodiments the targeting means includes a lectin or an antibody.

In yet further embodiments of the method the first neural cell is a neural progenitor cell. Further in such embodiments the originating region includes at least a portion of the subventricular zone, or the dentate gyrus in the hippocampus; and still additional embodiments the second region includes at least a portion of corpus callosum.

In several embodiments of the method wherein the first neural cell is a neural progenitor cell, the second region includes at least a portion of cortex or the second region includes a lesion.

In yet additional embodiments of the method the portion of a first neural cell includes an outgrowth of a neuronal cell, such as a dendrite, an axon or an axonal growth cone; in such embodiments the originating region may include at least a portion of a neurite tract from which outgrowth occurs, and the second region may include a lesion of the central nervous system.

In a second aspect the invention provides a method of use of a composition that is effective to treat or ameliorate a neurological pathology in a subject, wherein the use includes contacting a second neural cell with a composition that is effective to decorate a second neural cell with a hydrated polymer, and wherein the second neural cell is located substantially within a zone of interest containing at least one of an originating region, a second region, and a space between them.

In various embodiments of the use the hydrated polymer includes a polysaccharide or a derivative thereof.

In additional embodiments of the use the composition includes a polynucleotide encoding a polypeptide having activity that synthesizes a hydrated polymer bound to an external surface of the second neural cell, or a complement thereof. In further embodiments of the use the composition includes a polysialyltransferase, or any substance that is an ortholog thereof, a variant thereof, a fragment of any of the foregoing, a peptidomimetic, or a mimetic compound, that exhibits polysialyltransferase activity.

In still additional embodiments of the use the composition includes a conjugate that includes the hydrated polymer bound to a targeting means wherein the targeting means binds to a moiety of the external surface of the second neural cell.

In yet additional embodiments of the use the originating region contains a first neural cell or portion thereof, in certain embodiments the first neural cell is a neural progenitor cell; and in additional embodiments the portion of a first neural cell includes an outgrowth of a neuronal cell, such as a dendrite, an axon or an axonal growth cone.

In further embodiments the pathology is a CNS lesion, and in certain embodiments the lesion is the result of a stroke or a traumatic injury; in still additional embodiments the pathology is a CNS neurite tract lesion.

In still a further aspect the invention provides a conjugate that includes a hydrated polymer bound to a targeting means such that the targeting means binds to a moiety of the external surface of a neural cell. In various embodiments of the conjugate the targeting means includes a lectin or an antibody. In certain additional embodiments the hydrated polymer includes a polysaccharide or a derivative thereof.

In yet an additional aspect the invention provides a lentiviral vector that includes a polynucleotide encoding a polypeptide such that the polypeptide has a biological activity that synthesizes a hydrated polymer bound to a moiety on an external surface of a mammalian cell, or a complement thereof, additionally the vector enters the cell and expresses the polynucleotide therein.

In various embodiments of the vector the polypeptide includes a polysialyltransferase, an ortholog thereof, a variant thereof, or a fragment of any of them. In certain additional embodiments the mammalian cell is a neural cell.

In still further embodiments of the vector the moiety is a protein; in various additional embodiments the moiety is a neural cell adhesion molecule.

In yet an additional aspect the invention provides an isolated polynucleotide containing a nucleotide sequence chosen from among:
a) a nucleotide sequence encoding a variant of a wild type polysialyltransferase whose amino acid sequence is at least 90% identical to the wild type amino acid sequence;
b) a nucleotide sequence complementary to a nucleotide sequence given in a); or
c) a nucleotide sequence that is a fragment of any of the nucleotide sequences of a) or b);
wherein the encoded variant or fragment exhibits polysialyltransferase activity.

In still an additional aspect the invention provides an isolated polypeptide including an amino acid sequence that is a variant of a wild type polysialyltransferase wherein the sequence is at least 90% identical to the wild type amino acid sequence, or a fragment thereof; wherein the variant or fragment exhibits polysialyltransferase activity.

### BRIEF DESCRIPTION OF THE FIGURES

FIG. 1. Panel A. Maps of lentiviral TVA-specific transfer vector carrying GFP (control) or GFP-PST (EGFP-PST) fusion protein.

Panel B. Left, Western blot of extracts COS-7 cells transfected with the control vector (GFP) and with the EGFP-PST fusion vector (GFP-PST), probed with anti-GFP antibody. Right, Western blot of the same cells (Control, GFP-PST) and of the cell extract after digestion with endo N (GFP-PST+endo N) probed with anti-PSA antibody, in the region of molecular weight over about 150 kDa.

Panel C. Original color microscopic image showing PSA-immunostaining of COS-7 cells cotransfected with NCAM and either GFP (top row), GFP-PST (middle row), or GFP-PST cells after endo N treatment ((20 U/ml for 12 hours, bottom row). Immunostaining was with anti-GFP (left column, green) or anti-PSA (right column, red). (Scale bar: 50 μm).

Panel D. Original color microscopic image showing ex vivo brain slice from GFAP-TVA adult mice incubated in the presence of lentiviral vector (GFP, green stain), exhibiting specific infection of GFAP-positive cells (red). (Scale bar: 20 μm).

FIG. 2. GFP-PST viral vector-induced PSA expression on astrocytes in a GFAP-TVA transgenic mouse brain.

Panel A. Light microscopic image of a sagittal brain section. The line shows the path for injection of lentivirus. The box indicates the area of the CC analyzed in the present study (Magnification: 10×).

Panel B. Microscopic images of uninjected cells or cells 30 days after injection with either GFP (as control) or with GFP-PST fusion, detected by immunostaining with anti-GFP, anti-GFAP and anti-PSA. The long dashed lines show the injection route into the CC. The dotted line shows the border between the CC and the SVZ cortex. The asterisk at the lower right corner of the lower left panel indicates the SVZ. (Scale bar: 50 μm).

Panel C. Original color microscopic images of the CC in a GFAP-TVA transgenic mouse brain injected with the GFP-PST virus, detected with anti-GFAP (blue) and anti-PSA (red); the merged image is on the right. (Scale bar: 20 μm).

FIG. 3. Effect of ectopic PSA expression on progenitor migration into the corpus callosum.

Panel A. Microscopic images of immunostaining by anti-BrdU (left) and anti-nestin (right) in CC of GFP-PST-injected animals as compared to un-injected and GFP-injected control animals. The dashed lines mark the injection path into the CC and the asterisk indicates the SVZ. The dotted line shows the border between the SVZ and the CC. The asterisk indicates the SVZ. (Scale bar: 50 μm).

Panel B. Quantitative analysis of BrdU-positive SVZ progenitor cells that have migrated into the uninjected and the GFP- and GFP-PST-injected CC.

FIG. 4. Panel A. Microscopic image of immunohistochemical staining using anti-PSA after injection of GFP-PST virus into the brain (the dashed lines show the injection route whereas the dotted line shows the border between the CC and the cortex; m indicates meninges; scale bar: 50 μm).

Panel B. Microscopic image of immunohistochemical staining using anti-BrdU staining showing migrating SVZ cells in an animal injected with GFP-PST virus. The parallel lines mark the needle track, the horizontal line indicates the border between the CC and the cortex, the dashed line marks the border between SVZ and CC), and the asterisk indicates the SVZ (Scale bar: 50 μm).

FIG. 5. Panel A. Original color image of immunohistochemical staining by anti-BrdU (green, left) and anti-nestin (red, center), with merged images on the right. The SVZ-rostral migratory stream (RMS) region is located between the parallel lines. The arrow shows the direction of SVZ cell migration.

Panel B. Original color image of immunohistochemical imaging of BrdU- (left), nestin-positive (center), and merged (right) SVZ progenitors migrating into the CC. The line shows the border between the SVZ (*) and CC. (Scale bar: 50 μm).

FIG. 6. Fate of progenitors that migrate to the CC.

Panel A. Original color photomicrograph of immunohistochemical imaging of BrdU-positive cells (green) in the CC of animals treated with GFP-PST virus and probed as well for an early oligodendrocytic marker with anti-04 (red, left), and for mature oligodendrocyte markers with anti-CNPase (red, center) and anti-MAG (red, right). The arrows indicate processes of CNPase- and MAG-positive cells (scale bar: 20 μm).

Panel B. Original color photomicrograph of immunohistochemical imaging of BrdU-positive cells (green) in animals treated with GFP-PST virus and probed as well for the early postmitotic neuronal marker NeuN (red, left) and TH (tyrosine hydroxylase, red, right). (Scale bar: 20 μm).

Panel C. Quantitation of the percentage of BrdU-positive cells co-expressing CNPase and/or O4 (oligodendrocytic markers), NeuN (early postmitotic neurons) NSE or TH (mature neurons) in the mechanically lesioned CC of animals injected with PST virus.

Panel D. Schematic diagram summarizing the fate of progenitor cells migrating into the injected CC in GFP-PST-injected animals.

FIG. 7. Effect of injecting ibotenic acid into the cortex.

Panel A. Annexin-V immunostaining 3 weeks after injection of ibotenic acid or the carrier PBS into the cerebral cortex. The inset shows a higher magnification of the area in the box of the ibotenic acid-injected cortex (scale bar: 200 μm, inset 20 μm). The white line marks the cortical surface.

Panel B. PSA immunostaining after injection of ibotenic acid or the carrier PBS into the cerebral cortex (scale bar: 200 μm). The white line indicates the cortical surface. The parallel lines indicate the injection route.

FIG. 8. Effect of PSA overexpression on migration of progenitor cells after a chemically induced cortical lesion.

Panel A. Immunostaining with anti-BrdU and anti-nestin antibodies in PST-infected brains and control-infected brains. The thin parallel white lines indicate the injection track. (Scale bar: 200 μm) CC: corpus callosum; m: meninges.

Panel B. Quantitation of the number of BrdU/nestin-positive cells in the chemical cortical lesion PST-injected brains, and in uninjected and control-injected brains. Values are the mean±SEM of migrating cells counted in 5-7 40 μm brain slices.

FIG. 9. Differentiation of migrating progenitors in a chemically induced cortical lesion.

Panel A. An adult mouse brain injected with viral PST after receiving an ibotenic acid induced chemical lesion. i. Sagittal section of the brain. The white line shows the injection route, the box crossing the line shows a region in the injection epicenter, and the box on the left side next to the line shows a region outside the epicenter (magnification: 20×). ii. and iii. Original color photomicrographs. ii. NF-M stain is red, and BrdU stain is green. Left. NF-M and BrdU double stained cells lateral to the injection route (lesioned area but outside the epicenter). Right. NF-M and BrdU double stained cells in the epicenter of the injection. (Scale bar: ii: 20 μm). iii. Higher magnification of the insets in ii. (Scale bar: iii: 10 μm).

Panel B. Original color photomicrographs showing immunohistochemical staining of BrdU-positive cells migrating into the chemically lesioned cortex, stained with antibodies against NF-M, ChAT, and TH. (Scale bar: 20 μm).

Panel C. Percentage of BrdU-positive progenitors that migrated into the lesioned cortex and co-expressed CNPase, GFAP, NF-M, ChAT, GAD, and TH.

FIG. 10. Schematic diagrams summarizing heterotropic migration of SVZ progenitors into CNS lesions. SVZ progenitors are larger ovals in blue; PSA is shown as red wavy lines; oligodendrocytes are shown as green wavy lines.

Panel A. Control with chemical lesion alone.

Panel B. Left: Expression of PSA only on the surface of astrocytes. Right: Expression of PSA after induction of a chemical lesion induced by ibotenic acid (including apoptosis).

FIG. 11. Corticospinal tract (CST) lesion and scar formation. Top: Schematic drawing showing corticospinal axons projecting from the cerebral cortex all along the spinal cord. A section of the right corticospinal tract was performed around the 10th vertebra and the viral vector was locally injected at the time of lesion. Bottom: After the lesion the distal part of the axons degenerate and a non-permissive glial scar forms at the site of lesion.

FIG. 12. Original color photomicrographs, which include green areas from the fluorescence of GFP, whether as a holoprotein or as a domain of a fusion with PST, and red areas staining for PSA. The lower two panels include white-line outlines superimposed on selected red-stained areas, to indicate approximate regions of strong red color. The top panels show images of glial scars from control mice injected with GFP virus, and the bottom panels show glial scars from PST-GFP treated animals. (Magnification 100×).

FIG. 13. Original color photomicrographs, which include green areas from the fluorescence of GFP, whether as a holoprotein or as a domain of a fusion with PST, red areas showing staining for PSA, and blue staining for axons. Selected blue regions are outlined by solid white lines to enclose approximate regions of strong blue color. In the upper right panel dashed white-line outlines surround selected regions of strong red stain. The top panel shows a single image from a control section showing axons (blue) at the entrance of the glial scar, which is green from GFP. The arrow at the bottom of this panel indicates the rostro-distal axis. In the bottom set of four images, the selected channels showing staining for PSA (red), axons (blue), and GFP (green) are overlaid in various pairwise combinations. (Magnification 400×).

FIG. 14. Left panel: Slightly rotated three-dimensional reconstructions of superimposed 100 μm thick transverse sections of the CST located 0.4-0.5 mm distal from the lesion. The arrows point to axons in the field. (Magnification 400×). Right panel:

Quantitation of axon regeneration in spinal sections distal to the lesion.

### DETAILED DESCRIPTION OF THE INVENTION

The Sequence Listing appended hereto includes SEQ ID NOS:1-4 as follows:

| | | GenBank | SEQ ID NO: | SEQ ID NO: |
|---|---|---|---|---|
| Species | Gene ID | Acc. Nos. | (DNA) | (PROTEIN) |
| Gallus gallus | Polysialyl- | AF008194, | 1 | 2 |
| | transferase | AAB95120 | | |
| | ST8Sia IV | | | |
| Homo sapiens | Polysialyl- | L41680.1, | 3 | 4 |
| | transferase | AAC41775.1 | | |

The present invention relates broadly to methods and compositions that promote the stimulated, or directed, migration of neural progenitor cells, or portions of neural cells such as dendritic or axonal projections, from a region of origin to a second region and the subsequent differentiation according to the environment that the second region presents to the progenitor. In cases of therapeutic interest, the second region is the site of a pathology, trauma or a disease, and the differentiation of the relocated progenitor enhances treatment of the defect. The stimulated migration is accomplished in the invention by decorating cells in the central nervous system in a zone of interest with hydrated macromolecules such that the decorated cells, which ordinarily adhere avidly to one another, are believed to interact less strongly because of the presence of the macromolecules. It is intended that migration occur to, along, or within the zone of interest. Without being bound by theory it is believed that weakening the intercellular binding interactions by so decorating cells facilitates the migration of the progenitor cells, or the outgrowth of the neural cell projections. As noted, the migration and differentiation of the progenitors or neural cell projections contributes to treatment of several central nervous system pathological states. The invention additionally provides certain compositions that are useful in the methods of the invention to accomplish their objectives.

The Examples reported herein demonstrate the potential exhibited by certain embodiments of the methods and compositions provided herein. In a transgenic mouse model system it is shown that glial cells, made receptive to a directed viral vector by virtue of the transgenic transformation, incorporate and express a gene whose encoded polypeptide polysialylates the glial cells. The modified glial cells beneficially facilitate migration of progenitor cells from the subventricular zone to the corpus callosum (useful in treatment of a variety of demyelinating diseases), or to the site of an experimentally induced cortical lesion (useful in treatment of stroke or cortical trauma) or they facilitate neurite outgrowth of axonal projections from corticospinal neurons through the site of an induced corticospinal lesion, which is useful in treatment of any CNS neurite tract injury or lesion.

More generally the invention provides methods and compositions for inducing analogous migration of progenitors, and outgrowth of neurite projections, in a normal, nontransgenic (i.e., “wild type”) subject, including a nonhuman mammal, and a human.

As used herein the terms “neural” or “neural cell”, and similar terms and phrases, relate to any cell of a central nervous system. Furthermore, as used herein, the term “neuronal”, “neuronal cell”, “neuron”, and similar terms and phrases, relate to the cells of the central nervous system that perform the nervous system function of signal transmission. As used herein the term “glial”, or “glial cell”, and related terms and phrases relate generally to cells of the central nervous system other than neuronal cells. Nonlimiting examples of glial cells include astrocytes, oligodendrocytes, and microglia.

As used herein the term “originating region” and similar terms and phrases relate, first, to any of several region of the central nervous system in which progenitor cells arise, and second, any region of the central nervous system from which neurons project axonal outgrowths, growth cones, dendrites and the like. Nonlimiting examples of an origin of progenitor cells include the subventricular zone and the dentate gyrus in the hippocampus.

As used herein the term “neurite tract” and similar terms and phrases relate to a CNS tract that may include one or more of neurons, axonal projections of neurons, and dendritic projections of neurons. Outgrowth of neouronal cell projections may occur in any orientation within a neurite tract. A neurite tract may occur within, or span, the brain, the spinal cord, and offshoots of the spinal cord that lead to various nerves of the peripheral nervous system.

As used herein the terms “progenitor” and “precursor cell”, and similar terms and phrases relate to a relatively undifferentiated cell capable of differentiating to provide any of a number of various specialized cell types. The terms “progenitor” and “precursor cell” are considered synonymous herein.

As used herein the term “migration” and similar terms and phrases, when relating to progenitor cells, refers to movement or translocation of the entire cell from one locus to another. When relating to neurons, the term “migration” and similar terms and phrases refer to outgrowth of a neurite extension or a growth cone from a location proximal to the cell body of the neuron to a more distal location.

As used herein the term “hydrated”, and similar terms and phrases, when referring to a polymer, a macromolecule, a polysaccharide, and similar substances, relate to a polymer, a macromolecule, a polysaccharide, and similar substances that is sufficiently hydrophilic, i.e., bears a sufficient number of pendant hydrophilic groups, that the substance attracts water of solvation when suspended in an aqueous or physiological medium. Frequently such hydrated polymers, macromolecules, polysaccharides, and similar substances swell as a result of the hydration of their pendant groups. Without wishing to be bound by theory, it is believed in the context of the present invention that decorating the exterior surface of a neural cell with macromolecules that become hydrated in physiological milieu of the central nervous system weakens cell-cell interactions between them.

As used herein the term “polysialylation” and similar terms and phrases relates to the effect obtained as a result of the action of a substance or composition exhibiting polysialyltransferase (PST) activity. Any such composition or substance having PST activity is encompassed within the scope of the invention. An extensive nonlimiting disclosure of certain embodiments of substances exhibiting PST activity is presented herein. Any equivalent composition or substance as understood by a worker of skill in fields related to the present invention, including by way of nonlimiting example neurobiology, neurology, enzymology, physiology, cell biology, and the like, is included within the scope of the invention. A nonlimiting aspect of polysialyltransferase activity includes synthesis of a polysialic acid bound to a neural cell adhesion molecule.

As used herein the phrase “hydrated polymer transferase (HPT)”, “hydrated polymer transferase activity”, and similar terms and phrases relate generally to an activity that binds any hydrated polymer to an extracellular moiety of a second neural cell. Any polypeptide or mimetic exhibiting PST activity is included in the general term “hydrated polymer transferase”.

As used herein the phrase “neural cell adhesion molecule” and similar terms and phrases relates to any member of a family of protein molecules involved in cell-cell adhesion between neurons. This family is extensive for any given species. The phrase includes members of the family as they occur in any species of animal of interest in a particular context. Any protein molecule recognized by a worker of skill in fields related to the present invention, including by way of nonlimiting example neurobiology, neurology, physiology, cell biology, and the like, is included within the scope of a neural cell adhesion molecule.

As used herein the term “lectin” and similar terms and phrases relate to a protein that has an affinity for binding to a saccharide, an oligosaccharide, or a polysaccharide. A lectin binds without limitation a complex carbohydrate moiety that is a component of the exterior surface of a cell.

As used herein the term “decorate” and similar terms and phrases relate generally to binding a substance to a substrate such as a macromolecule, a supramolecular assembly, an organelle, or a cell surface. “Decorating” refers to binding a large amount of the substance to the substrate. By way of nonlimiting example, in the context of the present invention, “decorating” a neural cell signifies binding to an external cell surface thereof a plurality of hydrated macromolecules.

As used herein the term “treat” and similar terms and phrases, when employed in reference to a disease or pathological condition related to a PST-related process in a cell, relates to a ministration to a subject that acts to impede progression of the disease or condition or to begin an improvement in the subject, as evaluated, by way of nonlimiting example, by a diagnostic value for a test, or a systemic symptom of the disease or condition.

As used herein the term “ameliorate” and similar terms and phrases, when employed in reference to a disease or pathological condition related to a PST-related process in a cell, relates to a ministration to a subject that acts to reduce or essentially eliminate manifestations of the disease or condition in the subject. Such manifestations include, by way of nonlimiting example, a diagnostic value for a test, or a systemic symptom of the disease or condition.

PST or HPT Polynucleotides

As used herein the terms “nucleic acid” and “polynucleotide” and similar terms and phrases are considered synonymous with each other, and are used as conventionally understood by workers of skill in fields such as biochemistry, molecular biology, genomics, and similar fields related to the field of the invention. A polynucleotide employed in the invention may be single stranded or it may be a base paired double stranded structure, or even a triple stranded base paired structure. A polynucleotide may be a DNA, an RNA, or any mixture or combination of a DNA strand and an RNA strand, such as, by way of nonlimiting example, a DNA-RNA duplex structure. A polynucleotide and an “oligonucleotide” as used herein are identical in any and all attributes defined here for a polynucleotide except for the length of a strand. As used herein, a polynucleotide may be about 50 nucleotides or base pairs in length or longer, or may be of the length of, or longer than, about 60, or about 70, or about 80, or about 100, or about 150, or about 200, or about 300, or about 400, or about 500, or about 700, or about 1000, or about 1500, or about 2000 or about 2500, or about 3000, nucleotides or base pairs or even longer. An oligonucleotide may be at least 3 nucleotides or base pairs in length, and may be shorter than about 70, or about 60, or about 50, or about 40, or about 30, or about 20, or about 15, or about 10 nucleotides or base pairs in length. Both polynucleotides and oligonucleotides may be chemically synthesized by standard synthetic techniques, e.g., using an automated DNA synthesizer. Oligonucleotides may be used as probes.

As used herein “fragment” and similar words relate to portions of a nucleic acid, polynucleotide or oligonucleotide, or to portions of a protein or polypeptide, shorter than the full sequence of a reference. The sequence of bases, or the sequence of amino acid residues, remaining in a fragment may be unaltered from the sequence of the corresponding portion of the molecule from which it arose, or the fragment may include a sequence that is a variant of the corresponding portion of the parent molecule. As contemplated herein, a fragment of a nucleic acid or polynucleotide, such as an oligonucleotide, is 15 or more bases in length, or 16 or more, 17 or more, 18 or more, 21 or more, 24 or more, 27 or more, 30 or more, 50 or more, 75 or more, 100 or more bases in length, up to a length that is one base shorter than the full length sequence. Any fragment of a polynucleotide may be chemically synthesized and may be used as a probe.

As used herein and in the claims “nucleotide sequence”, “oligonucleotide sequence” or “polynucleotide sequence”, “polypeptide sequence”, “amino acid sequence”, “peptide sequence”, “oligopeptide sequence”, and similar terms, relate interchangeably both to the sequence of bases or amino acids that an oligonucleotide or polynucleotide, or polypeptide, peptide or oligopeptide has, as well as to the oligonucleotide or polynucleotide, or polypeptide, peptide or oligopeptide structure possessing the sequence. A nucleotide sequence or a polynucleotide sequence, or polypeptide sequence, peptide sequence or oligopeptide sequence furthermore relates to any natural or synthetic polynucleotide or oligonucleotide, or polypeptide, peptide or oligopeptide, in which the sequence of bases or amino acids is defined by description or recitation of a particular sequence of letters designating bases or amino acids as conventionally employed in the field.

Nucleotide residues occupy sequential positions in an oligonucleotide or a polynucleotide. Accordingly a modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

As used herein and in the claims, a “nucleic acid” or “polynucleotide”, and similar terms based on these, refer to polymers composed of naturally occurring nucleotides as well as to polymers composed of synthetic or modified nucleotides. Thus, as used herein, a polynucleotide that is a RNA, or a polynucleotide that is a DNA may include naturally occurring moieties such as the naturally occurring bases and ribose or deoxyribose rings, or they may be composed of synthetic or modified moieties as described in the following. The linkages between nucleotides is commonly the 3′-5′ phosphate linkage, which may be a natural phosphodiester linkage, a phosphothioester linkage, and still other synthetic linkages. Examples of modified backbones include, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates. Additional linkages include phosphotriester, siloxane, carbonate, carboxymethylester, acetamidate, carbamate, thioether, bridged phosphoramidate, bridged methylene phosphonate, bridged phosphorothioate and sulfone internucleotide linkages. Other polymeric linkages include 2′-5′ linked analogs of these. See U.S. Pat. Nos. 6,503,754 and 6,506,735 and references cited therein, incorporated herein by reference. The monosaccharide may be modified by being, for example, a pentose or a hexose other than a ribose or a deoxyribose. The monosaccharide may also be modified by substituting hydryoxyl groups with hydro or amino groups, by esterifying additional hydroxyl groups, and so on.

The bases in oligonucleotides and polynucleotides may be “unmodified” or “natural” bases include the purine bases adenine (A) and guanine (G), and the pyrimidine bases thymine (T), cytosine (C) and uracil (U). In addition they may be bases with modifications or substitutions. As used herein, modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-fluoro-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition (1991) 30, 613, and those disclosed by Sanghvi, Y. S., Chapter 15, Antisense Research and Applications, pages 289-302, Crooke, S. T. and Lebleu, B., ed., CRC Press, 1993. Certain of these bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications. See U.S. Pat. Nos. 6,503,754 and 6,506,735 and references cited therein, incorporated herein by reference.

Nucleotides may also be modified to harbor a label. Nucleotides bearing a fluorescent label or a biotin label, for example, are available from Sigma (St. Louis, Mo.).

As used herein an “isolated” nucleic acid molecule is one that is separated from at least one other nucleic acid molecule that is present in the natural source of the nucleic acid. Examples of isolated nucleic acid molecules include, but are not limited to, recombinant polynucleotide molecules, recombinant polynucleotide sequences contained in a vector, recombinant polynucleotide molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated PST or HPT nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A polynucleotide used in the present invention may include a sequence such as that of SEQ ID NO:1 or SEQ ID NO:3, encoding a polysialyltransferase given by SEQ ID NO:2 or SEQ ID NO:4, respectively. Alternatively, a polynucleotide for use in the invention may encode any wild type ortholog of a polysialyltransferase given by SEQ ID NO:2 or 4 or a variant thereof, or a fragment of any of them. In addition a polynucleotide used in the present invention may encode a polypeptide having activity that generally decorates a second neural cell with a hydrated polymer (i.e., hydrated polymer transferase activity, or HPT). A nucleic acid molecule employed in the present invention can be isolated using standard molecular biology techniques and the sequence information provided herein. Using all or a portion of the nucleic acid sequence of any such polynucleotide as a hybridization probe, PST or HPT nucleic acid sequences or sequences catalyzing the transfer of any hydrated polymer to a protein or other moiety on an exterior portion of the second neural cell can be isolated using standard hybridization and cloning techniques (e.g., as described in Sambrook et al., eds., MOLECULAR CLONING: A Laboratory Manual 3rd Ed., Cold Spring Harbor Laboratory Press, Cold Spring Harbor, N.Y., 2001; and Brent et al., Current Protocols in Molecular Biology, Wiley Interscience Publishers, (2003)).

As used herein, the term “complementary” refers to Watson-Crick or Hoogsteen base pairing between nucleotides units of a nucleic acid molecule. As used herein and in the claims, the term “complementary” and similar words, relate to the ability of a first nucleic acid base in one strand of a nucleic acid, polynucleotide or oligonucleotide to interact specifically only with a particular second nucleic acid base in a second strand of a nucleic acid, polynucleotide or oligonucleotide. By way of nonlimiting example, if the naturally occurring bases are considered, A and T or U interact with each other, and G and C interact with each other. As employed in this invention and in the claims, “complementary” is intended to signify “fully complementary” within a region, namely, that when two polynucleotide strands are aligned with each other, at least in the region each base in a sequence of contiguous bases in one strand is complementary to an interacting base in a sequence of contiguous bases of the same length on the opposing strand.

Detection and Labeling. A PST or HPT polynucleotide or a PST or HPT polypeptide may be detected in many ways. Detecting may include any one or more processes that result in the ability to observe the presence and or the amount of a PST or HPT polynucleotide or a PST or HPT polypeptide. In one embodiment a sample nucleic acid containing a PST or HPT polynucleotide may be detected prior to expansion. In an alternative embodiment a PST or HPT polynucleotide in a sample may be expanded to provide an expanded PST or HPT polynucleotide, and the expanded polynucleotide is detected or quantitated. Physical, chemical or biological methods may be used to detect and quantitate a PST or HPT polynucleotide. Physical methods include, by way of nonlimiting example, optical visualization including various microscopic techniques such as fluorescence microscopy, confocal microscopy, microscopic visualization of in situ hybridization, surface plasmon resonance (SPR) detection such as binding a probe to a surface and using SPR to detect binding of a PST or HPT polynucleotide or a PST or HPT polypeptide to the immobilized probe, or having a probe in a chromatographic medium and detecting binding of a PST or HPT polynucleotide in the chromatographic medium. Physical methods further include a gel electrophoresis or capillary electrophoresis format in which PST or HPT polynucleotides or PST or HPT polypeptides are resolved from other polynucleotides or polypeptides, and the resolved PST or HPT polynucleotides or PST or HPT polypeptides are detected. Physical methods additionally include broadly any spectroscopic method of detecting or quantitating a substance. Chemical methods include hybridization methods generally in which a PST or HPT polynucleotide hybridizes to a probe. Biological methods include causing a PST or HPT polynucleotide or a PST or HPT polypeptide to exert a biological effect on a cell, and detecting the effect. The present invention discloses examples of biological effects which may be used as a biological assay. In many embodiments, the polynucleotides may be labeled as described below to assist in detection and quantitation. For example, a sample nucleic acid may be labeled by chemical or enzymatic addition of a labeled moiety such as a labeled nucleotide or a labeled oligonucleotide linker. Many equivalent methods of detecting a PST or HPT polynucleotide or a PST or HPT polypeptide are known to workers of skill in fields related to the field of the invention, and are contemplated to be within the scope of the invention.

A nucleic acid of the invention can be expanded using cDNA, mRNA or alternatively, genomic DNA, as a template together with appropriate oligonucleotide primers according to any of a wide range of PCR amplification techniques. The nucleic acid so amplified can be cloned into an appropriate vector and characterized by DNA sequence analysis. Furthermore, oligonucleotides corresponding to PST or HPT nucleotide sequences can be prepared by standard synthetic techniques, e.g., using an automated DNA synthesizer.

Expanded polynucleotides may be detected and/or quantitated directly. For example, an expanded polynucleotide may be subjected to electrophoresis in a gel that resolves by size, and stained with a dye that reveals its presence and amount. Alternatively an expanded PST or HPT polynucleotide may be detected upon exposure to a probe nucleic acid under hybridizing conditions (see below) and binding by hybridization is detected and/or quantitated. Detection is accomplished in any way that permits determining that a PST or HPT polynucleotide has bound to the probe. This can be achieved by detecting the change in a physical property of the probe brought about by hybridizing a fragment. A nonlimiting example of such a physical detection method is SPR.

An alternative way of accomplishing detection is to use a labeled form of a PST or HPT polynucleotide or a PST or HPT polypeptide, and to detect the bound label. The polynucleotide may be labeled as an additional feature in the process of expanding the nucleic acid, or by other methods. A label may be incorporated into the fragments by use of modified nucleotides included in the compositions used to expand the fragment populations. A label may be a radioisotopic label, such as ¹²⁵I, ³⁵S, ³²P, ¹⁴C, or ³H, for example, that is detectable by its radioactivity. Alternatively, a label may be selected such that it can be detected using a spectroscopic method, for example. In one instance, a label may be a chromophore, absorbing incident light. A preferred label is one detectable by luminescence. Luminescence includes fluorescence, phosphorescence, and chemiluminescence. Thus a label that fluoresces, or that phosphoresces, or that induces a chemiluminscent reaction, may be employed. Examples of suitable fluorescent labels, or fluorochromes, include a ¹⁵²Eu label, a fluorescein label, a rhodamine label, a phycoerythrin label, a phycocyanin label, Cy-3, Cy-5, an allophycocyanin label, an o-phthalaldehyde label, and a fluorescamine label. Luminescent labels afford detection with high sensitivity. A label may furthermore be a magnetic resonance label, such as a stable free radical label detectable by electron paramagnetic resonance, or a nuclear label, detectable by nuclear magnetic resonance. A label may still further be a ligand in a specific ligand-receptor pair; the presence of the ligand is then detected by the secondary binding of the specific receptor, which commonly is itself labeled for detection. Nonlimiting examples of such ligand-receptor pairs include biotin and streptavidin or avidin, a hapten such as digoxigenin or antigen and its specific antibody, and so forth. A label still further may be a fusion sequence appended to a PST or HPT polynucleotide or a PST or HPT polypeptide. Such fusions permit isolation and/or detection and quantitation of the PST or HPT polynucleotide or a PST or HPT polypeptide. By way of nonlimiting example, a fusion sequence may be a FLAG sequence, a polyhistidine sequence, a fluorescent protein sequence such as a green fluorescent protein, a yellow fluorescent protein, an alkaline phosphatase, a glutathione transferase, and the like. In summary, labeling can be accomplished in a wide variety of ways known to workers of skill in fields related to the present disclosure. Any equivalent label that permits detecting and/or quantitation of a PST or HPT polynucleotide or a PST or HPT polypeptide is understood to fall within the scope of the invention.

Detecting, quantitating, including labeling, methods are known generally to workers of skill in fields related to the present invention, including, by way of nonlimiting example, workers of skill in spectroscopy, nucleic acid chemistry, biochemistry, molecular biology and cell biology. Quantitating permits determining the quantity, mass, or concentration of a nucleic acid or polynucleotide, or fragment thereof, that has bound to the probe. Quantitation includes determining the amount of change in a physical, chemical, or biological property as described in this and preceding paragraphs. For example the intensity of a signal originating from a label may be used to assess the quantity of the nucleic acid bound to the probe. Any equivalent process yielding a way of detecting the presence and/or the quantity, mass, or concentration of a polynucleotide or fragment thereof that hybridizes to a probe nucleic acid is envisioned to be within the scope employed in the present invention.

Variant PST or HPT Polynucleotides

The invention further encompasses nucleic acid molecules that differ from the disclosed PST or HPT nucleotide sequences. For example a sequence may differ due to degeneracy of the genetic code. These nucleic acids thus encode the same PST or HPT protein as that encoded by the nucleotide sequence shown in SEQ ID NO:1 or SEQ ID NO:3, have degenerate sequences that encode any ortholog of a polysialyltransferase. In such embodiments, an isolated nucleic acid molecule of the invention has a nucleotide sequence encoding a protein having an amino acid sequence shown in any of SEQ ID NO:2 or SEQ ID NO:4.

In addition to the PST or HPT nucleotide sequences shown in any of SEQ ID NO:1 or SEQ ID NO:3, it will be appreciated by those skilled in the art that DNA allelic sequence polymorphisms that lead to changes in the amino acid sequences of PST or HPT protein may exist within a population (e.g., the human population). Such natural allelic variations can typically result in 1-5% variance in the nucleotide sequence of the PST or HPT polynucleotide. Any and all such nucleotide variations and resulting amino acid polymorphisms in the PST or HPT protein that are the result of natural allelic variation and that do not alter the functional activity of the PST or HPT protein are intended to be within the scope of the invention. In general, a variant polynucleotide may encode an altered nucleotide sequence encoding a polypeptide that differs from a wild type polysialyltransferase by 1 or more amino acid residues, provided that the polypeptide retains polysialyltransferase activity.

Moreover, nucleic acid molecules encoding PST or HPT orthologs from other species, and thus that have a nucleotide sequence that differs from the human sequence of any of SEQ ID NO:1 or SEQ ID NO:3, are intended to be within the scope of the invention. Nucleic acid molecules corresponding to natural allelic variants and orthologs of the PST or HPT cDNAs of the invention can be isolated based on their homology to the human PST or HPT nucleic acids disclosed herein using the human cDNAs, or a portion thereof, as a hybridization probe according to standard hybridization techniques under stringent hybridization conditions.

Conservative Mutations

In addition to naturally-occurring allelic variants of the PST or HPT sequence that may exist in the population, the skilled artisan will further appreciate that variants of the nucleotide sequence of any of SEQ ID NO:1 or SEQ ID NO:3 or a polynucleotide encoding any orthologous polysialyltransferase can be generated by a skilled artisan, thereby leading to changes in the amino acid sequence of the encoded PST or HPT protein, without altering the functional activity of the PST or HPT protein. For example, nucleotide substitutions leading to amino acid substitutions at “non-essential” amino acid residues can be made in the sequence of any of SEQ ID NO:1 or SEQ ID NO:3. A “non-essential” amino acid residue is a residue at a position in the sequence that can be altered from the wild-type sequence of the PST or HPT protein without altering the biological activity of the resulting gene product, whereas an “essential” amino acid residue is a residue at a position that is required for biological activity. For example, amino acid residues that are invariant among members of a family of PST or HPT proteins, of which the PST or HPT proteins employed in the present invention are members, are predicted to be particularly unamenable to alteration. Whether a position in an amino acid sequence of a polypeptide is invariant or subject to substitution is readily apparent upon examination of a multiple sequence alignment of homologs, orthologs and paralogs of the polypeptide.

Thus an important aspect of the invention pertains to nucleic acid molecules encoding PST or HPT proteins that contain changes in amino acid residues that are not essential for activity. Such PST or HPT proteins differ in amino acid sequence from any of SEQ ID NO:2 or SEQ ID NO:4 or orthologous PST or HPT proteins yet retain biological activity. In one embodiment, the isolated nucleic acid molecule comprises a nucleotide sequence encoding a protein, wherein the protein comprises an amino acid sequence at least about 75% similar to the amino acid sequence of any of SEQ ID NO:2 or SEQ ID NO:4. Preferably, the protein encoded by the nucleic acid is at least about 80% identical to any of SEQ ID NO:2 or SEQ ID NO:4, more preferably at least about 85%, at least about 90%, at least about 95%, at least about 97%, at least about 98%, and most preferably at least about 99% identical to SEQ ID NO:2 or SEQ ID NO:4. An isolated nucleic acid molecule encoding a protein similar to the protein of any of SEQ ID NO:2 or SEQ ID NO:4 can be created by introducing one or more nucleotide substitutions, additions or deletions into the corresponding nucleotide sequence, such that one or more amino acid substitutions, additions or deletions are introduced into the encoded protein.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Certain amino acids have side chains with more than one classifiable characteristic, such as polar amino acid with a long aliphatic side chain. The amino acid families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, tryptophan, cysteine), nonpolar side chains (e.g., glycine, alanine, valine, leucine, isoleucine, proline, phenylalanine, methionine, tyrosine, tryptophan, lysine), beta-branched side chains (e.g., threonine, valine, isoleucine) aromatic side chains (e.g., tyrosine, phenylalanine, tryptophan, histidine) and metal-complexing side chains (e.g., aspartic acid, glutamic acid, asparagine, glutamine, serine, threonine, tyrosine, cysteine, methionine and histidine). Mutations can be introduced into SEQ ID NO:1 or SEQ ID NO:3 by standard techniques, such as site-directed mutagenesis and PCR-mediated mutagenesis. Alternatively, in another embodiment, mutations can be introduced randomly along all or part of a PST or HPT coding sequence, such as by saturation mutagenesis, and the resultant mutants can be screened for PST or HPT protein biological activity to identify mutants that retain activity. Following mutagenesis of SEQ ID NO:1 or SEQ ID NO:3 the encoded protein can be expressed by any recombinant technology known in the art and the activity of the protein can be determined.

Determining Similarity Between Two or More Sequences

To determine the percent similarity of two amino acid sequences or of two nucleic acids, the sequences are aligned for optimal comparison purposes (e.g., gaps can be introduced in either of the sequences being compared for optimal alignment between the sequences). As used herein amino acid or nucleotide “identity” is synonymous with amino acid or nucleotide “homology”.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T or U, C, G, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. In polypeptides the “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

“Identity,” as known in the art, is a relationship between two or more polypeptide sequences or two or more polynucleotide sequences, as determined by, comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk. A. M., ed., Oxford University Press, New York, 1988; Biocomputing: Informatics and Genome Projects, Smith, D. W., ed., Academic Press, New York, 1993; Computer Analysis of Sequence Data, Part I. Griffin, A. M., and Griffin, H. G., eds. Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press. New York, 1991; and Carillo, H., and Lipman, D., SIAM J. Applied Math. (1988) 48: 1073. Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devercux, J., et al. (1984) Nucleic Acids Research 12(1): 387), BLASTP, BLASTN, and FASTA (Atschul, S. F. et al. (1990) J. Molec. Biol. 215: 403-410. The BLAST X program is publicly available from NCBI and other sources (BLAST Manual, Altschul, S., et al., NCBI NLM NIH Bethesda, Md. 20894; Altschul, S., et al. (1990) J. Mol. Biol. 215: 403-410. The well known Smith Waterman algorithm may also be used to determine identity.

Additionally the BLAST alignment tool is useful for detecting similarities and percent identity between two sequences. BLAST is available on the World Wide Web at the National Center for Biotechnology Information site. References describing BLAST analysis include Madden, T. L., Tatusov, R. L. & Zhang, J. (1996) Meth. Enzymol. 266:131-141; Altschul, S. F., Madden, T. L., Schäffer, A. A., Zhang, J., Zhang, Z., Miller, W. & Lipman, D. J. (1997) Nucleic Acids Res. 25:3389-3402; and Zhang, J. & Madden, T. L. (1997) Genome Res. 7:649-656.

Polypeptides

As used herein the term “protein”, “polypeptide”, or “oligopeptide”, and similar words based on these, relate to polymers of alpha amino acids joined in peptide linkage. Alpha amino acids include those encoded by triplet codons of nucleic acids, polynucleotides and oligonucleotides. They may also include amino acids with side chains that differ from those encoded by the genetic code.

As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

As used herein an “amino acid” designates any one of the naturally occurring alpha-amino acids that are found in proteins. In addition, the term “amino acid” designates any nonnaturally occurring amino acids known to workers of skill in protein chemistry, biochemistry, and other fields related to the present invention. These include, by way of nonlimiting example, sarcosine, hydroxyproline, norleucine, alloisoleucine, cyclohexylalanine, phenylglycine, homocysteine, dihydroxyphenylalanine, ornithine, citrulline, D-amino acid isomers of naturally occurring L-amino acids, and others. In addition an amino acid may be modified or derivatized, for example by coupling the side chain with a label. Any amino acid known to a worker of skill in the art may be incorporated into a polypeptide disclosed herein.

The term “epitope tagged” when used herein refers to a chimeric polypeptide comprising a PST or HPT polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the terms “active” or “activity” and similar terms refer to form(s) of a polypeptide which retain a biological and/or an immunological activity of any native or naturally-occurring PST or HPT ortholog, wherein “biological” activity includes polysialyltransferase activity. Additionally a PST or HPT polypeptide has the ability to induce the production of an antibody against an antigenic epitope possessed by a native or naturally-occurring PST or HPT and an “immunological” activity refers to the ability to induce the production of an antibody against an antigenic epitope possessed by a native or naturally-occurring PST.

PST or HPT Proteins and Polypeptides

The invention relates to an isolated PST or HPT protein whose sequence is provided in any of SEQ ID NO:2 or SEQ ID NO:4, or to any ortholog thereof. The invention also includes a mutant or variant protein any of whose residues may be changed from the corresponding residue of SEQ ID NO:2 or SEQ ID NO:4 or of any ortholog thereof while still encoding a protein that maintains its PST or HPT protein-like activities and physiological functions, or a functional fragment thereof. For example, the invention includes the polypeptides encoded by the variant PST or HPT nucleic acids described above. In the mutant or variant protein, up to 20% or more of the residues may be so changed.

In general, a PST or HPT protein-like variant that preserves PST or HPT activity includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a non-essential or conservative substitution as defined above. Furthermore, without limiting the scope of the invention, one or more positions of any of SEQ ID NO:2 or SEQ ID NO:4 or orthologs thereof may be substituted such that a mutant or variant protein may include one or more substitutions.

The invention also includes isolated PST or HPT proteins, and biologically active portions thereof, or derivatives, fragments, analogs or homologs thereof. Also provided are polypeptide fragments suitable for use as immunogens to raise anti-PST protein antibodies. A fragment of a protein or polypeptide, such as a peptide or oligopeptide, may be 5 amino acid residues or more in length, or 6 or more, 7 or more, 8 or more, 9 or more, 10 or more, 15 or more, 20 or more, 25 or more, 30 or more, 50 or more, 10 or more residues in length, up to a length that is one residue shorter than the full length sequence. In one embodiment, native PST or HPT proteins can be isolated from cells or tissue sources by an appropriate purification scheme using standard protein purification techniques. In another embodiment, PST or HPT proteins are produced by recombinant DNA techniques. Alternative to recombinant expression, a PST or HPT protein or polypeptide can be synthesized chemically using standard peptide synthesis techniques. Purification of proteins and polypeptides is described, for example, in texts such as “Protein Purification, 3^{rd} Ed.”, R. K. Scopes, Springer-Verlag, New York, 1994; “Protein Methods, 2^{nd} Ed.,” D. M. Bollag, M. D. Rozycki, and S. J. Edelsterin, Wiley-Liss, New York, 1996; and “Guide to Protein Purification”, M. Deutscher, Academic Press, New York, 2001.

Biologically active portions of a PST or HPT protein include peptides comprising amino acid sequences sufficiently similar to or derived from the amino acid sequence of the PST or HPT protein, e.g., the amino acid sequence shown in SEQ ID NO:2 or SEQ ID NO:4 that include fewer amino acids than the full length PST or HPT proteins, and exhibit at least one activity of a PST or HPT protein. Typically, biologically active portions comprise a domain or motif with at least one activity of the PST or HPT protein. A biologically active portion of a PST or HPT protein can be a polypeptide which is, for example, 10, 25, 50, 100 or more amino acids in length.

A biologically active portion of a PST or HPT protein employed in the present invention may contain at least one of the above-identified domains conserved among the PST or HPT family of proteins. Moreover, other biologically active portions, in which other regions of the protein are deleted, can be prepared by recombinant techniques and evaluated for one or more of the functional activities of a native PST or HPT protein.

In an embodiment, the PST protein has an amino acid sequence shown in any of SEQ ID NO:2 or SEQ ID NO:4 or an ortholog thereof. In other embodiments, the PST protein is a variant that is substantially similar to any of SEQ ID NO:2 or SEQ ID NO:4 or an ortholog thereof and retains the PST activity of any of SEQ ID NO:2 or SEQ ID NO:4 or their orthologs, yet differs in amino acid sequence due to natural allelic variation or to imposed mutagenesis, as described in detail below. Accordingly, in another embodiment, the PST or HPT protein is a protein that comprises an amino acid sequence at least about 45% similar, and more preferably about 55% or more, 65% or more, 70% or more, 75% or more, 80% or more, 85% or more, 90% or more, 95% or more, 98% or more, or even 99% or more similar to the amino acid sequence of any of SEQ ID NO:2 or SEQ ID NO:4 and retains the functional activity of the PST or HPT proteins of the corresponding polypeptide having the sequence of SEQ ID NO:2 or SEQ ID NO:4 or orthologs thereof. Nonlimiting examples of particular amino acid residues that may changed in a variant polypeptide molecule are identified as the result of an alignment of a PST or HPT polypeptide with a homologous or paralogous polypeptide.

Chimeric and Fusion Proteins

The invention also provides PST or HPT protein chimeric or fusion proteins. As used herein, a PST or HPT protein “chimeric protein” or “fusion protein” includes a PST or HPT polypeptide operatively linked to a non-PST polypeptide. A “PST polypeptide” refers to a polypeptide having an amino acid sequence corresponding to the PST or HPT protein, whereas a “non-PST polypeptide” refers to a polypeptide having an amino acid sequence corresponding to a protein that is not substantially similar to the PST or HPT protein, e.g., a protein that is different from the PST or HPT protein and that is derived from the same or a different organism. Within a fusion protein containing a PST or HPT protein the PST or HPT polypeptide can correspond to all or a portion of a PST or HPT protein. In one embodiment, a PST or HPT protein fusion protein comprises a full length PST or HPT protein or at least one biologically active fragment of a PST or HPT protein. In another embodiment, a PST or HPT protein fusion protein comprises at least two fragments of a PST or HPT protein each of which retains its biological activity. Within the fusion protein, the term “operatively linked” is intended to indicate that the PST or HPT polypeptide and the non-PST or non-HPT polypeptide are fused in-frame to each other. The non-PST polypeptide can be fused to the N-terminus or C-terminus of the PST or HPT polypeptide.

In another embodiment, the fusion protein is a GST-PST protein fusion protein in which the PST or HPT protein sequences are fused to the C-terminus of the GST (i.e., glutathione S-transferase) sequences. Such fusion proteins can facilitate the purification of recombinant PST or HPT protein. Additional fusion embodiments include FLAG-tagged fusions and fluorescent protein fusions, useful for purification and detection of the fusion construct.

In yet another embodiment, the fusion protein is a PST or HPT protein containing a heterologous signal sequence at its N-terminus. For example, the native PST or HPT protein signal sequence can be removed and replaced with a signal sequence from another protein. In certain host cells (e.g., mammalian host cells), expression and/or secretion of the PST or HPT protein can be increased through use of a heterologous signal sequence.

A PST or HPT protein chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Brent et al., Current Protocols in Molecular Biology, Wiley Interscience Publishers, (2003)). Moreover, many expression vectors are commercially available that already encode a fusion moiety (e.g., a GST polypeptide). A PST or HPT protein-encoding nucleic acid can be cloned into such an expression vector such that the fusion moiety is linked in-frame to the PST or HPT protein.

A “specific binding agent” of a PST or HPT polypeptide or a PST or HPT oligopeptide is any substance that specifically binds the PST or HPT polypeptide or oligopeptide, but binds weakly or not at all to other polypeptides and oligopeptides. Nonlimiting examples of specific binding agents include antibodies, specific receptors for PST or HPT polypeptides, binding domains of such antibodies and receptors, aptamers, imprinted polymers, and so forth.

PST or HPT Mimetics

The present invention also pertains to fragments or variants of the PST or HPT proteins that function as PST or HPT protein agonists (mimetics). An mimetic of the PST or HPT protein can retain substantially the same, or a subset of, the PST or HPT activities of the naturally occurring form of the PST or HPT protein.

Variants or fragments of the PST or HPT protein that function as agonists (mimetics) can be identified by screening combinatorial libraries of mutants, e.g., truncation mutants, of the PST or HPT protein for PST or HPT protein agonist activity. In one embodiment, a variegated library of PST or HPT variants is generated by combinatorial mutagenesis at the nucleic acid level and is encoded by a variegated gene library. A variegated library of PST or HPT variants can be produced by, for example, enzymatically ligating a mixture of synthetic oligonucleotides into gene sequences such that a degenerate set of potential PST or HPT sequences is expressible as individual polypeptides, or alternatively, as a set of larger fusion proteins (e.g., for phage display) containing the set of PST or HPT sequences therein. Chemical synthesis of a degenerate gene sequence can be performed in an automatic DNA synthesizer, and the synthetic gene then ligated into an appropriate expression vector. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

As the PST or HPT gene family contains a critical region of high conservation, peptide mimetics of PST or HPT proteins would also be predicted to act as PST or HPT modulators. Such peptides are derived or designed from PST or HPT family proteins that block PST or HPT function. These mimetics are expected to block function of all the highly related PST or HPT proteins. Suitable peptide mimetics to PST or HPT proteins can be made according to conventional methods based on an understanding of the regions in the polypeptides required for PST or HPT protein activity. Briefly, a short amino acid sequence is identified in a protein by conventional structure-function studies such as deletion or mutation analysis of the wild-type protein, as well as by multisequence alignment. The amino acid sequence of the peptide mimetic may be composed of amino acids matching this region in whole or in part. Such amino acids could be replaced with other chemical structures resembling the original amino acids but imparting pharmacologically better properties, such as higher inhibitory activity, stability, half-life or bioavailability.

Polypeptide Libraries

In addition, libraries of fragments of the PST or HPT protein coding sequence can be used to generate a variegated population of functional fragments for screening and subsequent selection of variants of a PST or HPT protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PST or HPT proteins. Recrusive ensemble mutagenesis (REM), a new technique that enhances the frequency of functional mutants in the libraries, can be used in combination with the screening assays to identify PST or HPT variants (Arkin and Yourvan (1992) Proc. Natl. Acad. Sci. USA 89:7811-7815; Delgrave et al. (1993) Protein Engineering 6:327-331).

Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species. Any antibody disclosed herein binds “immunospecifically” to its cognate antigen. By immunospecific binding is meant that an antibody raised by challenging a host with a particular immunogen binds to a molecule such as an antigen that includes the immunogenic moiety with a high affinity, and binds with only a weak affinity or not at all to non-immunogen-containing molecules. As used in this definition, high affinity means having a dissociation constant less than about 1×10⁻⁶ M, and weak affinity means having a dissociation constant higher than about 1×10⁻⁶ M.

An isolated protein employed in the invention intended to serve as an antigen, or a portion or fragment thereof, can be used as an immunogen to generate antibodies that immunospecifically bind the antigen, using standard techniques for polyclonal and monoclonal antibody preparation. In several embodiments of the invention an antibody that specifically binds an exterior surface component of a second neural cell is employed as a targeting means to deliver a conjugate to the surface of the second neural cell. In these embodiments the conjugate includes the targeting antibody bound to a hydrated polymer described herein. An immunogen may include a composition derived from the second neural cell. For example a full-length cell surface protein can be used or, alternatively, antigenic peptide fragments may serve as immunogens. An antigenic peptide fragment comprises at least 6 amino acid residues of the amino acid sequence of the full length protein as well as other proteins or polypeptides employed herein, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

1. Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor.

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

2. Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). Alternatively, the lymphocytes can be immunized in vitro. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor: J. Immunol., 133:3001 (1984); Brodeur et al.: Monoclonal Antibody Production Techniques and Applications, Marcel Dekker, Inc., New York, (1987) pp. 51-63).

Monoclonal antibodies secreted by various clones or subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567.

3. Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature 321:522-525 (1986); Riechmann et al., Nature, 332:323-327 (1988); Verhoeyen et al., Science 239:1534-1536 (1988)), by substituting rodent CDRs or CDR sequences for the corresponding sequences of a human antibody. (See also U.S. Pat. No. 5,225,539.) The humanized antibody optimally also will comprise at least a portion of an immunoglobulin constant region (Fc), typically that of a human immunoglobulin (Jones et al., 1986; Riechmann et al., 1988; and Presta (1992) Curr. Op. Struct. Biol., 2:593-596).

4. Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al. (1983) Immunol Today 4: 72) and the EBV hybridoma technique to produce human monoclonal antibodies (see Cole, et al. (1985) In: MONOCLONAL ANTIBODIES AND CANCER THERAPY, Alan R. Liss, Inc., pp. 77-96). Human monoclonal antibodies may be utilized in the practice employed in the present invention and may be produced by using human hybridomas (see Cote, et al. (1983) Proc Natl Acad Sci USA 80: 2026-2030) or by transforming human B-cells with Epstein Barr Virus in vitro (see Cole, et al. (1985) In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter (1991) J. Mol. Biol., 227:381; Marks et al. (1991) J. Mol. Biol., 222:581). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (1992) (Bio/Technology 10, 779-783); Lonberg et al. ((1994) Nature 368 856-859); Morrison ((1994) Nature 368, 812-13); Fishwild et al, ((1996) Nature Biotechnology 14, 845-51); Neuberger ((1996) Nature Biotechnology 14, 826); and Lonberg and Huszar ((1995) Intern. Rev. Immunol. 13 65-93). A method for producing an antibody of interest, such as a human antibody, is also disclosed in U.S. Pat. No. 5,916,771.

Human antibodies may additionally be produced using transgenic nonhuman animals which are modified so as to produce fully human antibodies rather than the animal's endogenous antibodies in response to challenge by an antigen. (See publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications.

5. F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab)2} fragment produced by pepsin digestion of an antibody molecule; (ii) an F_{ab} fragment generated by reducing the disulfide bridges of an F(ab)₂ fragment; (iii) an F_{ab} fragment generated by the treatment of the antibody molecule with papain and a reducing agent and (iv) F, fragments.

6. Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al. (1991) EMBO J., 10:3655-3659. For further details of generating bispecific antibodies see, for example, Suresh et al. (1986) Methods in Enzymology, 121:210.

PST- or HPT-Containing Recombinant Vectors and Host Cells

Another aspect of the invention pertains to use of vectors, preferably expression vectors, containing a nucleic acid encoding a PST or HPT protein, or derivatives, fragments, orthologs and variants thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, lentiviruses, adenoviruses and adeno-associated viruses), which serve equivalent functions. An example of a lentiviral transfer vector is disclosed in U.S. Pat. No. 6,790,657.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to a regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel (1990) GENE EXPRESSION TECHNOLOGY: METHODS IN ENZYMOLOGY 185, Academic Press, San Diego, Calif. Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., PST or HPT proteins, mutant forms of the PST or HPT protein, fusion proteins, etc.).

The recombinant expression vectors of the invention can be designed for expression of the PST or HPT protein in prokaryotic or eukaryotic cells. For example, the PST or HPT protein can be expressed in bacterial cells such as E. coli, insect cells (using baculovirus expression vectors) yeast cells or mammalian cells. Suitable host cells are discussed further in Goeddel (1990). Alternatively, the recombinant expression vector can be transcribed and translated in vitro, for example using T7 promoter regulatory sequences and T7 polymerase.

Promoter regions can be selected from any desired gene using vectors that contain a reporter transcription unit lacking a promoter region, such as a chloramphenicol acetyl transferase (“CAT”), or the luciferase (LUC) transcription unit, downstream of restriction site or sites for introducing a candidate promoter fragment; i.e., a fragment that may contain a promoter. For example, introduction into the vector of a promoter-containing fragment at the restriction site upstream of the CAT or LUC gene engenders production of CAT or LUC activity, respectively, which can be detected by standard CAT or LUC assays. Vectors suitable to this end are well known and readily available. Two such vectors are pKK232-8 and pCM7. Thus, promoters for expression of polynucleotides employed in the present invention include not only well-known and readily available promoters, but also promoters that readily may be obtained by the foregoing technique, using a reporter gene.

Expression of proteins in prokaryotes is most often carried out in E. coli with vectors containing constitutive or inducible promoters directing the expression of either fusion or non-fusion proteins. Among known bacterial promoters suitable for expression of polynucleotides and polypeptides are the E. coli lacI and lacZ promoters, the T3 and T7 promoters, the T5 tac promoter, the lambda PR, PL promoters and the trp promoter. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al. (1990) GENE EXPRESSION TECHNOLOGY: METHODS IN ENZYMOLOGY 185, Academic Press, San Diego, Calif. 60-89).

In another embodiment, the PST or HPT expression vector is a yeast expression vector. Examples of vectors for expression in yeast S. cerivisae include pYepSec I (Baldari, et al., (1987) EMBO J 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the PST or HPT protein can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. Other eukaryotic promoters include the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

For other suitable expression systems for both prokaryotic and eukaryotic cells. See, e.g., Sambrook et al., MOLECULAR CLONING: A LABORATORY MANUAL. 3rd ed., Cold Spring Harbor Laboratory, Cold Spring Harbor Laboratory Press, Cold Spring Harbor, N.Y., 2001.

In another embodiment, the recombinant mammalian expression vector is capable of directing expression of the nucleic acid preferentially in a particular cell type. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a PST or HPT mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. For a discussion of the regulation of gene expression using antisense genes see Weintraub et al., “Antisense RNA as a molecular tool for genetic analysis,” Reviews—Trends in Genetics, Vol. 1(1) 1986.

The invention further provides a vector such as a viral vector that infects a second neural cell and expresses a polynucleotide therein encoding a polypeptide that decorates an extracellular domain of a protein of the cell with a hydrated polymer. Selectivity of infection or expression is accomplished by including on the virion a moiety that binds a target on the second neural cell, or that includes a regulatory sequence that responds to regulatory effectors found within the second neural cell.

Host Cells

Another aspect of the invention pertains to preparing or use of host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the PST or HPT protein can be expressed in bacterial cells such as E. coli, insect cells, yeast or mammalian cells (such as Chinese hamster ovary cells (CHO) or COS cells). Other suitable host cells are known to those skilled in the art.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and WI38 cells, neuronal stem cells and astrocytes.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (2001), Brent et al. (2003), and other laboratory manuals.

For stable transfection of mammalian cells, in order to identify and select stable integrants, a gene that encodes a selectable marker (e.g., resistance to antibiotics) is generally introduced into the host cells along with the gene of interest. Various selectable markers include those that confer resistance to drugs, such as G418, hygromycin and methotrexate.

Cell Culture

A cell culture to express PST or HPT is propagated using standard culture conditions. 24 hours before transfection, at approx. 80% confluency, the cells are trypsinized and diluted 1:5 with fresh medium without antibiotics (1-3×10⁵ cells/ml) and transferred to 24-well plates (500 ml/well). Transfection is performed using a commercially available lipofection kit or by FuGENE6 or by electroporation, calcium phosphate particle incorporation, or ballistic particles, and PST or HPT expression is monitored using standard techniques with positive and negative control. A positive control is cells that naturally express PST or HPT while a negative control is cells that do not express PST or HPT.

A host cell of the invention, such as a prokaryotic or eukaryotic host cell in culture, can be used to produce (i.e., express) the PST or HPT protein. Accordingly, the invention further provides methods for producing the PST or HPT protein using the host cells of the invention. In one embodiment, the method comprises culturing the host cell of invention (into which a recombinant expression vector encoding the PST or HPT protein has been introduced) in a suitable medium such that the PST or HPT protein is produced. In another embodiment, the method further comprises isolating the PST or HPT protein from the medium or the host cell.

Transgenic Animals

The host cells of the invention can also be used to produce nonhuman transgenic animals. For example, in one embodiment, a host cell of the invention is a fertilized oocyte or an embryonic stem cell into which any protein-coding sequences of interest have been introduced, or from which they have been knocked out. Such host cells can then be used to create non-human transgenic animals in which exogenous protein sequences have been introduced into their genome or homologous recombinant animals in which endogenous protein sequences have been altered. Such animals are useful for studying the function and/or activity of PST or HPT proteins and for identifying and/or evaluating modulators of PST or HPT protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous PST or HPT gene has been altered by homologous recombination between the endogenous gene and an exogenous DNA molecule introduced into a cell of the animal, e.g., an embryonic cell of the animal, prior to development of the animal.

A transgenic animal can be created by introducing a protein-encoding nucleic acid into the male pronuclei of a fertilized oocyte, e.g., by microinjection, retroviral infection, and allowing the oocyte to develop in a pseudopregnant female foster animal. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. A transgenic founder animal can then be used to breed additional animals carrying the transgene. Moreover, transgenic animals carrying a transgene encoding a protein of interest in the methods disclosed herein can further be bred to other transgenic animals carrying other transgenes. See e.g., Thomas et al. (1987) Cell 51:503 for a description of homologous recombination vectors. The vector is introduced into an embryonic stem cell line (e.g., by electroporation) and cells in which the introduced protein gene has homologously recombined with the endogenous protein gene are selected (see e.g., Li et al. (1992) Cell 69:915). See e.g., Bradley 1987, In: TERATOCARCINOMAS AND EMBRYONIC STEM CELLS: A PRACTICAL APPROACH, Robertson, ed. IRL, Oxford, pp. 113-152, and Bradley (1991) Curr Opin Biotechnol 2:823-829; PCT International Publication Nos.: WO 90/1184; WO 91/01140; WO 92/0968; and WO 93/04169.

Pharmaceutical Compositions

The pharmaceutical compositions disclosed herein are useful for preventing, treating or ameliorating pathological conditions related to demyelinating diseases, stroke, cortical trauma, corticospinal trauma, or any other neurodegenerative disorder where loss of certain CNS cell type occur. Moreover, the pharmaceutical composition disclosed herein are to be administered to a patient at therapeutically effective doses. A therapeutically effective dose refers to that amount of the compound sufficient to result in substantially inhibiting the development of, the treatment of, or the amelioration of said conditions.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The PST or HPT nucleic acid molecules, PST or HPT proteins, conjugates, and other compositions of the invention, and derivatives, fragments, analogs and homologs thereof are designated “active compounds” or “therapeutics” herein. These therapeutics can be incorporated into pharmaceutical compositions suitable for administration to a subject. Such compositions typically comprise the nucleic acid molecule, protein, or antibody and a pharmaceutically acceptable carrier.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in textbooks such as Remington's Pharmaceutical Sciences, Gennaro A R (Ed.) 20^{th} edition (2000) Williams & Wilkins Pa., USA, and Wilson and Gisvold's Textbook of Organic Medicinal and Pharmaceutical Chemistry, by Delgado and Remers, Lippincott-Raven., which are incorporated herein by reference. Preferred examples of components that may be used in such carriers or diluents include, but are not limited to, water, saline, phosphate salts, carboxylate salts, amino acid solutions, Ringer's solutions, dextrose solution, and 5% human serum albumin. Liposomes and non-aqueous vehicles such as fixed oils may also be used.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for the rectum.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods.

Microencapsulation of recombinant proteins for sustained release has been successfully performed with human growth hormone (rhGH), interferon-(rhIFN-), interleukin-2, and MN rgp120. Johnson et al., Nat Med. 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York. 1995), pp. 439-462; WO 97/03692, WO 96/40072, WO 96/7399; and U.S. Pat. No. 5,654,010.

The nucleic acid molecules of the invention can be inserted into vectors and used as gene therapy vectors. Gene therapy vectors may include antisense polynucleotides and inhibitory polynucleotides including microRNA (miRNA), modified miRNA, small inhibitory RNA (si RNA), and modified siRNA, wherein modifications are introduced as described in this invention at least to confer stability on the molecules. In one embodiment of gene therapy a PST or HPT nucleic acid is part of an expression vector that expresses a PST protein or an HPT protein or fragment or chimeric protein thereof in a subject. In particular, such a nucleic acid has a promoter operably linked to the PST or HPT coding region, said promoter being inducible or constitutive, and, optionally, tissue-specific. In another particular embodiment, a nucleic acid molecule is used in which the PST or HPT coding sequences and any other desired sequences are flanked by regions that promote homologous recombination at a desired site in the genome, thus providing for intrachromosomal expression of a PST or HPT nucleic acid (Koller and Smithies, 1989, Proc. Natl. Acad. Sci. USA 86:8932-8935; Zijlstra et al., 1989, Nature 342:435-438).

Gene therapy vectors can be delivered to a subject by any of a number of routes, e.g., as described in U.S. Pat. No. 5,703,055, by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. Delivery can thus also include, e.g., intravenous injection, local administration (see U.S. Pat. No. 5,328,470) or stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

In certain embodiments of the invention a lentiviral vector incorporating a sequence encoding a polypeptide with PST or HPT activity may be used. Lentiviral vectors of use in the invention typically exhibit a broad range of infectivity, and infect at least cells that are quiescent, i.e., not actively dividing.

In alternative embodiments a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The PST or HPT nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient. In a preferred embodiment, the cell used for gene therapy is autologous to the patient.

Dosages and desired drug concentrations of pharmaceutical compositions employed in the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PST or HPT polypeptide is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. No. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously, intramuscularly, or directly within the CNS) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC50 (i.e., the concentration of the test compound that achieves a half-maximal inhibition of symptoms). Such information can then be used to determine useful doses and routes for administration in humans.

A therapeutically effective dose refers to that amount of active ingredient useful to prevent, treat or ameliorate a particular pathological condition of interest. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies are used in formulating a range of dosage for human use. The dosage contained in such compositions is preferably within a range of circulating concentrations that include the ED50 with little or no toxicity. The dosage varies within this range depending upon the dosage-form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

Methods of Treatment

The present invention provides for therapeutic methods of treating a neurological disorder, such as a pathology that can benefit from stimulated migration of a neural cell or a portion thereof to a region where the pathology is expressed. These include a subject suffering from stroke, a cerebral trauma, a corticospinal trauma, and related pathologies.

Diseases and disorders that are characterized by demyelination or diseases that are characterized by loss of certain types of CNS cells, and related pathologies, may be treated with therapeutics that increase activity of PST or HPT or that increase the expression of PSA-NCAM on the exterior surface of a neural cell. Therapeutics that upregulate activity may be administered in a therapeutic manner. Therapeutics that may be utilized include, but are not limited to, a PST or HPT polynucleotide, a PST or HPT polypeptide, a PST or HPT peptide, or analogs, derivatives, fragments or homologs thereof, a conjugate of a moiety that targets a component of a neural cell surface and a hydrated macromolecule, or an agonist that increases bioavailability.

Polysialyltransferase Polynucleotides and Polypeptides

In various embodiments the present invention employs polysialyltransferase (PST) polynucleotides and the polypeptides encoded by them. PST polynucleotides may be isolated, characterized and prepared, by way of nonlimiting example, by methods such as those described herein. A polynucleotide encoding chicken (Gallus gallus) polysialyltransferase mRNA (GenBank Acc. No. AF008194) is shown in Table 1.

TABLE 1

(SEQ ID NO: 1)

| | |
|---|---|
| 1 | atgcgttccg tcaggaagag gtggactgtg tgcaccataa gtctcctcct catcttctac |
| 61 | aagactaagg agatcgcgcg cactgaggag cgccaggagg ctccgctcgc cggagatggt |
| 121 | gagctgagtt tgagtcgatc aatgatcaat agctctgata aaataatccg aaagggcggc |
| 181 | tccgctatct tccagcattc tgtagaaggt tggaaaatca attctacttt ggtactggag |
| 241 | ataagaaaga gtattctccg attcttggat gcggaaagag atgtctcagt ggtcaagagc |
| 301 | agctttaagc caggagatgt aatccattat gtactagaca gacgtcgcac cctaaatatt |
| 361 | tctcaqgatt tgcacagcct tctccctgag gtttccccaa tgaagaatcg ccgatttaaa |
| 421 | acctgtgctg tagttggaaa ttctggcatc cttctggaca gcgggtgtgg aaaagagatt |
| 481 | gatacccatg attttgttat aaggtgcaat ctagctcccg tggtggagtt tgctgcagat |
| 541 | gtgggaaata aatctgattt tattaccatg aacccatcag ttgtacaaag agcatttgga |
| 601 | ggctttcgga atgagagtga cagagaaaaa tttgggcata gactatccat gctgaatgac |
| 661 | agtgtccttt ggatccctgc tttcatggtc aaaggcggag agaagcattt ggagtgggtt |
| 721 | aatgcattaa tccttaagaa taaattgaaa gtgcgaaccg cctatccatc actgagactt |
| 781 | attcatgctg tcagaggtta ctggctcaca aacaaggtcc acatcaaacg acccagcact |
| 841 | ggtctcctca tgtacacgct tgccaccaga ttctgtgatg aaattcacct gtatggattt |
| 901 | tggccattcc caaaggattt acatggaaaa ccagtcaaat atcattatta cgatgatctg |
| 961 | aagtatcggt acttttctaa tgccagccct cacaggatgc cattagagtt taaaacatta |
| 1021 | tatgtattac ataacagagg agcacttaag ttaacaacgg gcaagtgcgt gaagcaataa |

In the sequence shown in Table 1, the entire sequence of 1080 bases encodes the PST polypeptide.

The polypeptide sequence predicted for chicken PST based on the nucleotide sequence of Table 1 is shown in Table 2 (GenBank Acc. No. AAB95120).

TABLE 2

(SEQ ID NO:2)

| | |
|---|---|
| 1 | mrsvrkrwtv ctislllify ktkeiartee rqeaplagdg elslsrsmin ssdkiirkgg |
| 61 | saifqhsveg wkinstlvle irksilrfld aerdvsvvks sfkpgdvihy vldrrrtlni |
| 121 | sqdlhsllpe vspmknrrfk tcavvgnsgi lldsgcgkei dthdfvircn lapvvefaad |
| 181 | vgnksdfitm npsvvqrafg gfrnesdrek fghrlsmlnd svlwipafmv kggekhlewv |
| 241 | nalilknklk vrtaypslrl ihavrgywlt nkvhikrpst gllmytlatr fcdeihlygf |
| 301 | wpfpkdlhgk pvkyhyyddl kyryfsnasp hrmplefktl yvlhnrgalk lttgkcvkq |

A polynucleotide encoding human PST (GenBank Acc. No. L41680.1) is shown in Table 3.

TABLE 3

(SEQ ID NO:3)

| | |
|---|---|
| 1 | cgcaaacagg gcgagaggtc gctgggcagc gttcgaggac cagagggagc tcggccacag |
| 61 | aagaccccag tgatctgatc ccgggatccc ggctccaagc tctcctcgca ttttacagat |
| 121 | ttcacccccg cgactatctc cccaaaacgg agcctttata tcaagagaag gtgcgggagc |
| 181 | tggggcaacc aggactttct cgggcaccca agatgcgctc cattaggaag aggtggacga |
| 241 | tctgcacaat aagtctgctc ctgatctttt ataagacaaa agaaatagca agaactgagg |
| 301 | agcaccagga gacgcaactc atcggagatg gtgaattgtc tttgagtcgg tcacttgtca |
| 361 | atagctctga taaaatcatt cgaaaggctg gctcttcaat cttccagcac aatgtagaag |
| 421 | gttggaaaat caattcctct ttggtcctag agataaggaa gaacatactt cgtttcttag |
| 481 | atgcagaacg agatgtgtca gtggtcaaga gcagttttaa gcctggtgat gtcatacact |
| 541 | atgtgcttga caggcgccgg acactaaaca tttctcatga tctacatagc ctcctacctg |
| 601 | aagtttcacc aatgaagaat cgcaggttta agacctgtgc agttgttgga aattctggca |
| 661 | ttctgttaga cagtgaatgt ggaaaggaga ttgacagtca caattttgta ataaggtgta |
| 721 | atctagctcc tgtggtggag tttgctgcag atgtgggaac taaatcagat tttattacca |
| 781 | tgaatccatc agttgtacaa agagcatttg gaggctttcg aaatgagagt gacagagaaa |
| 841 | aatttgtgca tagactttcc atgctgaatg acagtgtcct ttggattcct gctttcatgg |
| 901 | tcaaaggagg agagaagcac gtggagtggg ttaatgcatt aatccttaag aataaactga |
| 961 | aagtgcgaac tgcctatccg tcattgagac ttattcatgc tgtcagaggt tactggctga |
| 1021 | ccaacaaagt tcctatcaaa agacccagca caggtcttct catgtataca cttgccacaa |
| 1081 | gattctgtga tgaaattcac ctgtatggat tctggccctt ccctaaggat ttaaatggaa |
| 1141 | aagcggtcaa atatcattat tatgatgact taaaatatag gtacttttcc aatgcaagcc |
| 1201 | ctcacagaat gccattagaa ttcaaaacat taaatgtgct acataataga ggagctctaa |
| 1261 | aactgacaac aggaaagtgt gtaaagcaat aaagcacatt ttgaaacaaa caatatgcac |
| 1321 | ttcttttctg agatgcttcc gaagatttga aaataggatc caaaacacgg ctgggtttca |
| 1381 | gcatccacca atgaactgaa aggtgaataa aggacgttca tgagaaatcg actaccagct |
| 1441 | gatgaaatac ctgcaaagtg ctctaaaaat taaatatttt gactttaagg gtcctagtaa |
| 1501 | gtgccacttc cactaagaat acagtttgaa tgtataatca gtagtgttta caagatccaa |
| 1561 | cagtgcactc atcattagtt aacaaagcaa atatgttcat cactgtcagg ctgcccacag |
| 1621 | caacaccaag catattagaa gaggaacccc aggaacgcaa ctcagacctt gggaaattaa |
| 1681 | accatccttg tcagcagaag ccaagatgga agcagtttga gcaatgaaat ccgtaagatt |
| 1741 | aaacaactca agtaaatgct tcagtcagga ctctgagtct gatcatgaat tttatgtttt |
| 1801 | aatttatgtt tttttttttg tcttctggaa tctcttttgg tttggatatt gggatgctta |
| 1861 | gaaatccttt ctgagatgca tatgagtgag gaaa |

In the sequence shown in Table 3, the coding sequence extends from position 213 to position 1292.

The polypeptide sequence predicted for the human PST protein based on the nucleotide sequence of Table 3 is shown in Table 4 (GenBank Acc. No. AAC41775.1).

TABLE 4

(SEQ ID NO:4)

| | |
|---|---|
| 1 | mrsirkrwti ctislllify ktkeiartee hqetqligdg elslsrslvn ssdkiirkag |
| 61 | ssifqhnveg wkinsslvle irknilrfld aerdvsvvks sfkpgdvihy vldrrrtlni |
| 121 | shdlhsllpe vspmknrrfk tcavvgnsgi lldsecgkei dshnfvircn lapvvefaad |
| 181 | vgtksdfitm npsvvqrafg gfrnesdrek fvhrlsmlnd svlwipafmv kggekhvewv |
| 241 | nalilknklk vrtaypslrl ihavrgywlt nkvpikrpst gllmytlatr fcdeihlygf |
| 301 | wpfpkdlngk avkyhyyddl kyryfsnasp hrmplefktl nvlhnrgalk lttgkcvkq |

As used herein and in the claims, the terms “PST polynucleotide”, and similar terms and phrases, relate generally to the nucleotide sequences shown in Tables 1 and 3, or their complements, as well as to an ortholog thereof, a variant polynucleotide encoding a variant polypeptide whose amino acid sequence is at least 80% identical, or at least 85% identical, or at least 90% identical, or at least 95% identical, or at least 97% identical, or at least 98% identical, or at least 99% identical, to an amino acid sequence given in Tables 2 and 4 or an ortholog thereof; or to a nucleotide sequence that is a fragment of any of the nucleotide sequences described in this paragraph; to a polynucleotide that is complementary to any of the foregoing polynucleotides; or to a nucleotide sequence that hybridizes to any nucleotide sequence described in this paragraph. Orthologous PST polynucleotides include, by way of nonlimiting example, murine PST ST8SiaII (GenBank Acc. No. NM_{—}009181) and murine PST ST8SiaIV (GenBank Acc. No. AJ223956). A PST polynucleotide additionally relates to sequence encoding a peptide that is a peptidomimetic of any of the above encoded polypeptides. In addition a PST polynucleotide refers to a polynucleotide described above in this paragraph wherein the aforesaid fragment encodes a mature form of the polypeptide. A PST polynucleotide further relates to a polynucleotide described above in this paragraph, or its complement, wherein a variant amino acid residue in the encoded polypeptide is a non-essential or a conservative substitution. A PST polynucleotide further relates to a polynucleotide encoding a fusion protein, wherein the polynucleotide comprises a first PST polynucleotide as described above in this paragraph and a second polynucleotide encoding a second polypeptide fused to the 5′ end or to the 3′ end of the first polynucleotide.

A PST polynucleotide employed in the invention additionally relates to a nucleic acid molecule that is a complement of the nucleotide sequence shown in any of SEQ ID NO:1 or SEQ ID NO:3, or a portion of this nucleotide sequence, or an ortholog thereof, or a variant of any of them. A nucleic acid molecule that is complementary to the nucleotide sequence shown in any of SEQ ID NO: 1 or SEQ ID NO:3 is one that is sufficiently complementary to the nucleotide sequence shown in of any of SEQ ID NO:1 or SEQ ID NO:3 that it hydrogen bonds no mismatches to the nucleotide sequence shown in of any of SEQ ID NO:1 or SEQ ID NO:3, thereby forming a stable duplex. A PST polynucleotide employed in the invention also relates to any polynucleotide that hybridizes under appropriate conditions of stringency to any of the PST polynucleotides described above.

As used herein and in the claims, the terms “PST polypeptide”, and similar terms and phrases relate generally to the amino acid sequences shown in Tables 2 and 4, as well as an ortholog thereof, or to a variant polypeptide whose amino acid sequence is at least 80% identical, or at least 85% identical, or at least 90% identical, or at least 95% identical, or at least 97% identical, or at least 98% identical, or at least 99% identical, to an amino acid sequence given in Tables 2 and 4; or to a polypeptide whose amino acid sequence is a fragment of any of the amino acid sequences described in this paragraph. Orthologous PST polypeptides include, by way of nonlimiting example, murine PST ST8SiaII (GenBank Acc. No. NM_{—}009181) and murine PST ST8SiaIV (GenBank Acc. No. AJ223956). A PST polypeptide additionally relates to a peptide that is a peptidomimetic of any of the above compositions. In addition a PST polypeptide refers to a polypeptide described above in this paragraph wherein the aforesaid fragment includes a mature form of the polypeptide. A PST polypeptide further relates to variant polypeptide described above in this paragraph, wherein a variant amino acid residue in the encoded polypeptide is a non-essential or a conservative substitution. A PST polypeptide further relates to a polypeptide encoding a fusion protein, wherein the polypeptide comprises a first PST polypeptide as described above in this paragraph and a second polypeptide encoding a second polypeptide fused to the N-terminal end or to the C-terminal end of the first polypeptide. All PST polypeptides employed in the invention exhibit polysialyltransferase activity.

Hydrated Macromolecules

The invention encompasses binding any of a large number of hydrated macromolecules to the exterior surface of a neural cell. In certain embodiments a hydrated macromolecule may be a hydrogel. As used herein a “hydrogel” and similar terms and phrases relate to a gel constituted of one or more hydrophilic polymers, i.e., polymers bearing pendant residues having a hydrophilic character. Such polymers crosslink with one another by noncovalent interactions, for example by hydrogen bond formation between polymer molecules. In addition a hydrogel imbibes large quantities of water when suspended in an aqueous medium, such that a large proportion of the volume of the hydrogel is occupied by water. Any polymer known to workers of skill in fields related to the present invention to form a hydrogel is comprehended within the scope of the invention. Any polymer with properties equivalent to those of a hydrogel suitable for use in the invention is likewise comprehended within the scope of the invention. Any such equivalent hydrogel will facilitate migration of a progenitor from an originating region to a region of interest.

A hydrated macromolecule may be a polysaccharide, such as a polysialic acid, a derivative of a polysialic acid such as a higher acyl radical bound to the amino group, a hyaluronic acid or derivative thereof, a polylactosamine or derivative thereof, a soluble cellulose derivative such as carboxymethylcellulose, an agarose, a chitosan, derivatives thereof, and the like. Additionally a hydrated macromolecule may be a polyethylene glycol or ester or other derivative thereof, a polyethylene oxide or derivative thereof, a polypropylene oxide or derivative thereof, a polyvinylpyrrolidone or derivative thereof, as well as any other hydrated macromolecule. Any equivalent hydrated macromolecule known to workers of skill in fields related to the present invention may be employed. Any such equivalent hydrated macromolecule will facilitate migration of a progenitor from an originating region to a region of interest.

A hydrated macromolecule may be bound either directly to a component of the cell surface such as a protein, a glycoprotein, a lipid, or a glycolipid, or it may be bound indirectly via a conjugate of the invention.

Conjugates

Conjugates of a hydrated macromolecule and a targeting moiety may be used to decorate the exterior surface of a cell in the practice of the invention. One class of targeting moiety consists of antibody molecules. Antibodies may be raised that are immunospecific for a component of the exterior surface of a cell, such as a membrane protein, a membrane glycoprotein, a glycolipid, and so forth. The antigen may be a peptide component of the membrane protein, or it may be a saccharide epitope of a glycoprotein or glycolipid. Immunization may be carried out with relatively purified components such as those listed, or a suspension of cell membrane or fragments thereof may be used. Polyclonal antibodies may be adequate to serve as the targeting moieties of the invention; alternatively monoclonal antibodies may be identified and raised for this purpose. Additionally, in certain applications, such as with demyelinating diseases, it may be advantageous to provide humanized antibodies or fully human antibodies as the targeting moiety. Antibodies are described extensively elsewhere in the present disclosure.

A second class of targeting moiety consists generally of lectins. Lectins are proteins with a binding affinity for specific polysaccharide groupings. Lectins are found in both plants and animals. Animal lectins, and especially human lectins, may be employed to advantage in the present invention. Lectins may be prepared by protein purification methods, or by recombinant methods such as those described herein, as well as by the peptide synthetic chemistry methods described herein. Such methods of preparing proteins are widely known to workers of skill in fields related to the present invention.

Conjugates are prepared by synthetic chemistry procedures that are widely practiced in fields related to the present invention. As an example, a hydrated macromolecule that is a polysaccharide may be activated for reaction with the amine groups of a protein by periodate oxidation of vic-diols of the saccharide residues, then forming a Schiffs base with the amine groups of the protein, and finally reduction with a borohydride reagent. As a second example, a carboxyl group of a hydrated macromolecule such as PSA may be reacted with the amine groups of a protein by activation with a carbodiimide reagent, or by using an activated ester such as an N-hydroxysuccinimide (reagents available from Pierce Chemical Co., Rockford, Ill.). As a third example a hydrated macromolecule may be activated or derivatized to permit it to react with one moiety of a bifunctional linker, and the second moiety of the linker is reacted with a group on a protein such as an amino group, a carboxyl group, or a sulfhydryl group (a wide range of bifunctional linkers is available from Pierce Chemical Co.).

Administration of Compositions of the Invention

Any of the compositions of the invention is to be administered to a subject in order to promote migration of progenitors from their originating region to a distant region of interest. Thus the composition may include a polynucleotide or vector encoding a polypeptide having PST or HPT activity. Alternatively, it may include a polypeptide, variant, fragment, mimetic or peptidomimetic that has PST or HPT activity. Still alternatively, it may include any conjugate of the invention as described herein. In order to effect the decoration of neural cells to an optimal extent, a mode of administration is to be used that optimizes delivery of the composition to the region of interest in the CNS.

An optimal way of achieving this is direct physical delivery to the region of interest. Thus it is contemplated that neurosurgical procedures are to be used to introduce the composition by means of a needle or catheter, either within the brain, or into the spinal cord, as the circumstances merit. An alternative procedure may involve catheterization to bring a catheter close to or directly to a region of interest within the brain or other portion of the CNS. Frequently such procedures are enhanced by use of radiological visualization during the course of the procedure to guide the placement of the needle or the catheter.

Systemic delivery of a composition having PST or HPT activity is also contemplated, wherein the circulatory system brings the composition to the region of interest. Examples of pharmaceutical compositions that may be used for systemic delivery are described herein. This may be advantageous in the case of conjugates targeting a moiety specific for neural cells.

### EXAMPLES

### Materials and Methods

A. Expression of Polysialic Acid in Astrocytes

Selective expression of polysialic acid (PSA) in astrocytes was achieved in transgenic GFAP-TVA mice that express the TVA receptor (Bates et al., 1993; Young et al., 1993) under control of the glial fibrillary acidic protein (GFAP) promoter (Holland and Varmus, 1998). The mice were infected with a TVA-specific vector HIV (ALSV-A) virus (Lewis et al., 2001) that harbors the ST8Sia IV/PST gene coding for the PSA-synthesizing enzyme polysialyltransferase (PST) (Angata and Fukuda, 2003). The TVA-specific vector recognizes and infects only cells expressing the TVA receptor. In the case of the GFAP-TVA mice the only cells expressing the TVA receptor are astrocytes; the vector infects specifically only these cells.

B. Insertion of GFP-PST into a Lentiviral Vector

cDNA encoding either enhanced green fluorescent protein (GFP) or chicken polysialyltransferase ST8Sia IV (GenBank Acc. No. AF008194, SEQ ID NO:1) fused to GFP (GFP-PST) (Canger and Rutishauser, 2004) was inserted into a pCS-CG transfer lentiviral plasmid (Miyoshi et al., 1998), between its unique NheI and XhoI cutting sites. The QIAGEN plasmid maxi Kit and the QIAprep Spin Miniprep Kit were used for DNA preparations, while the Rapid DNA Ligation Kit and the QIACK gel extraction Kit were used for DNA ligation and purification respectively. All kits were from Qiagen, CA.

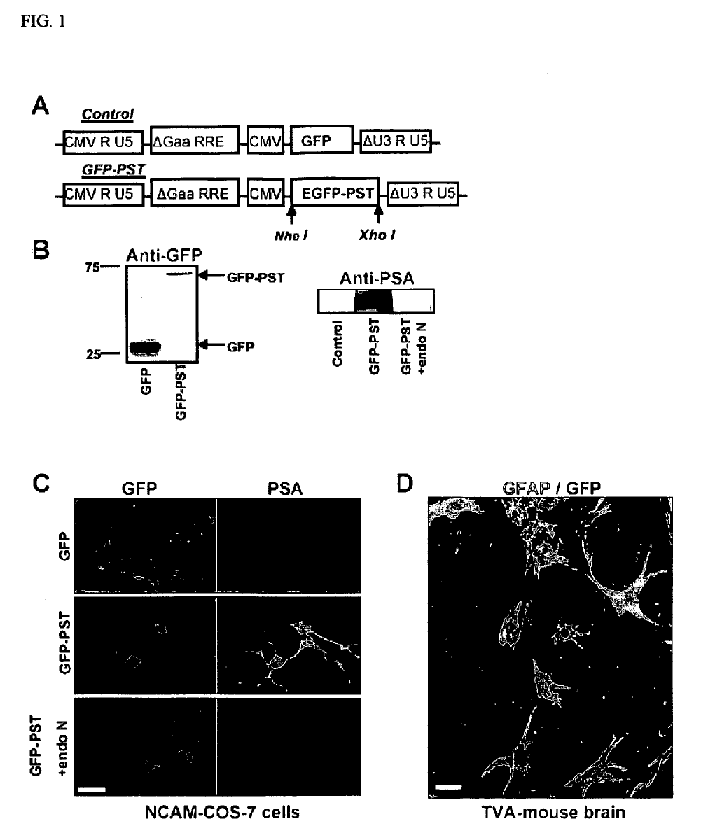

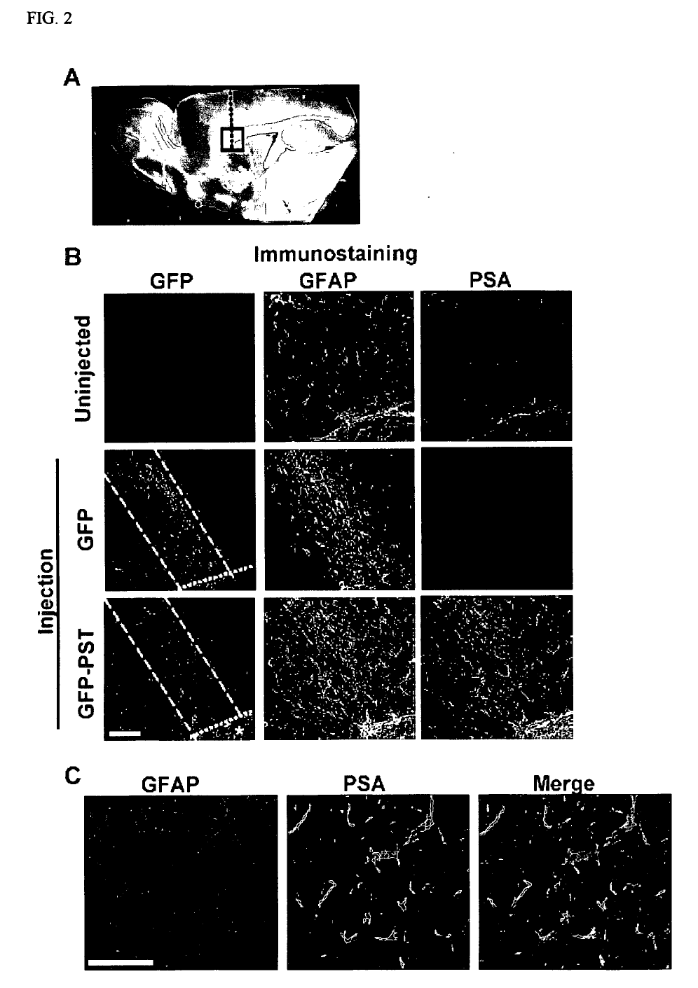

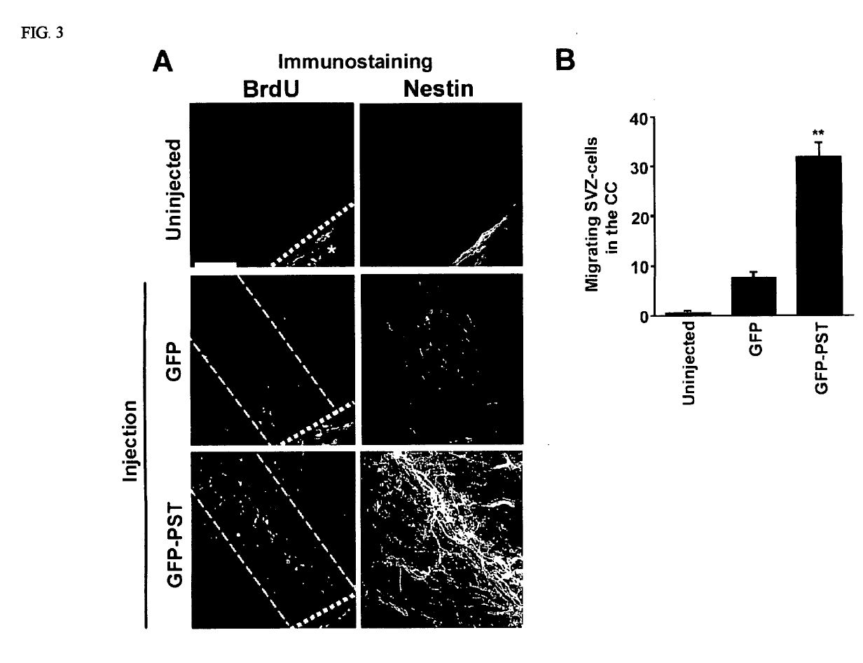

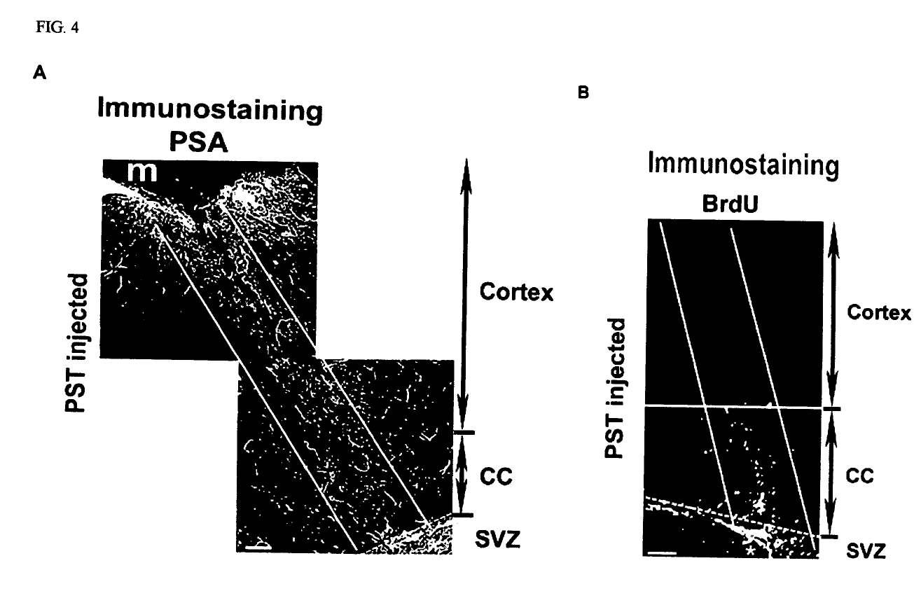

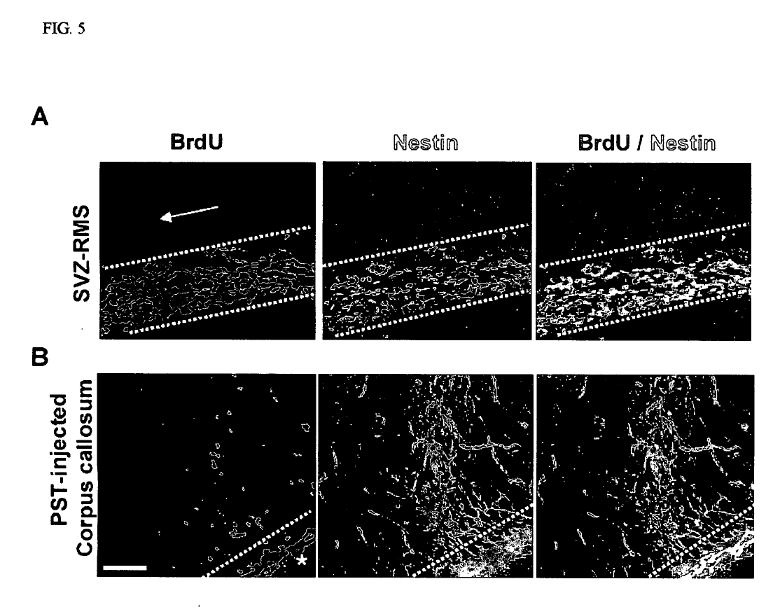

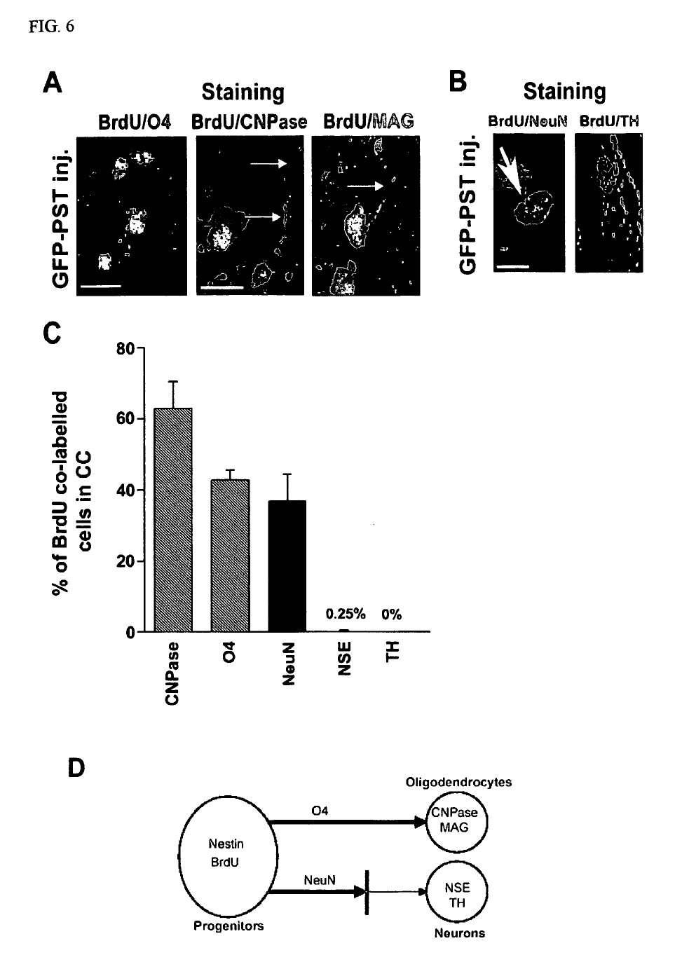

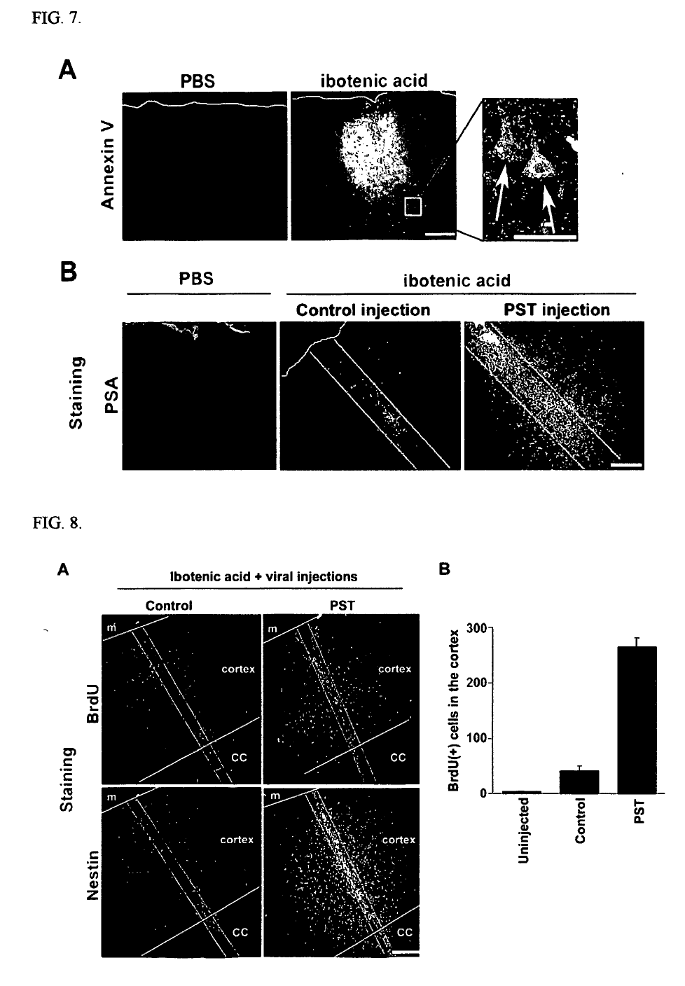

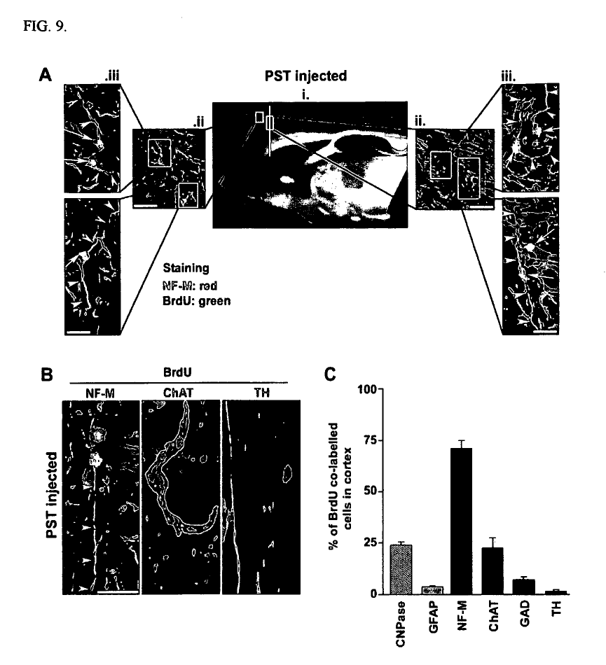

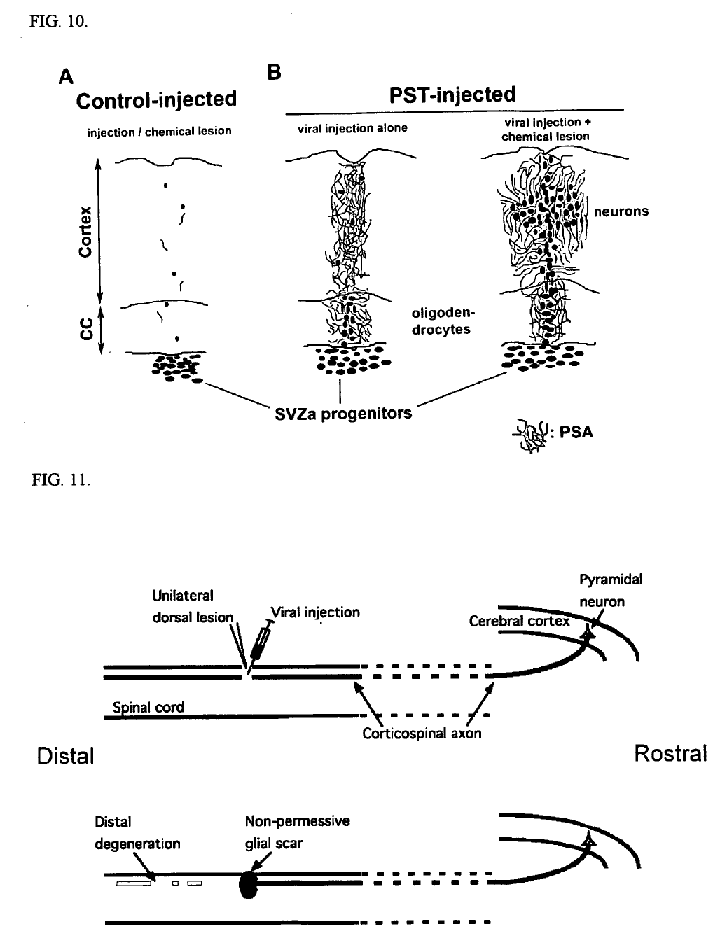

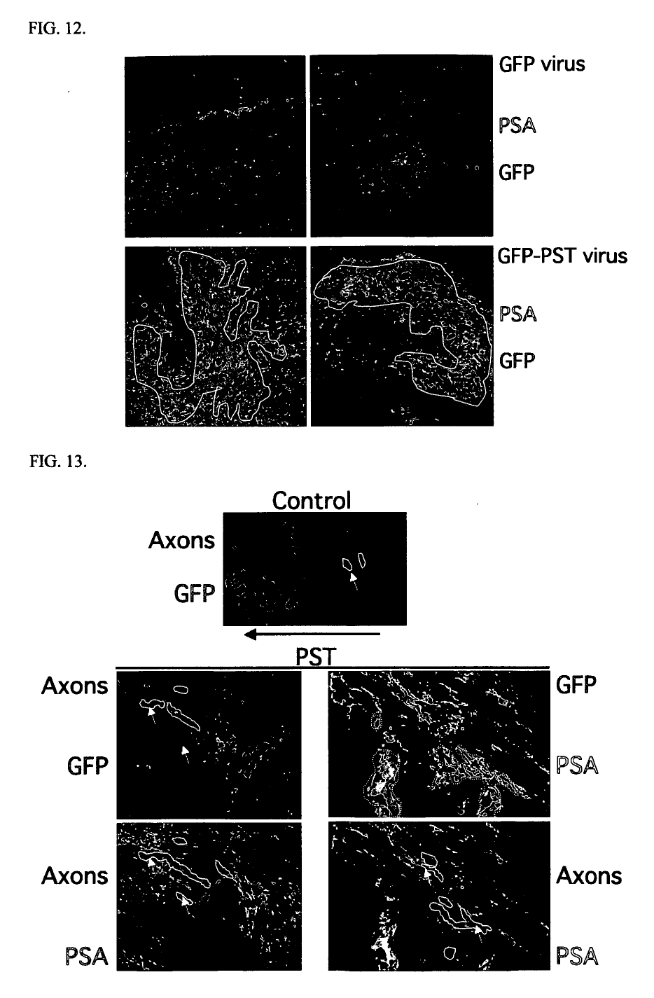

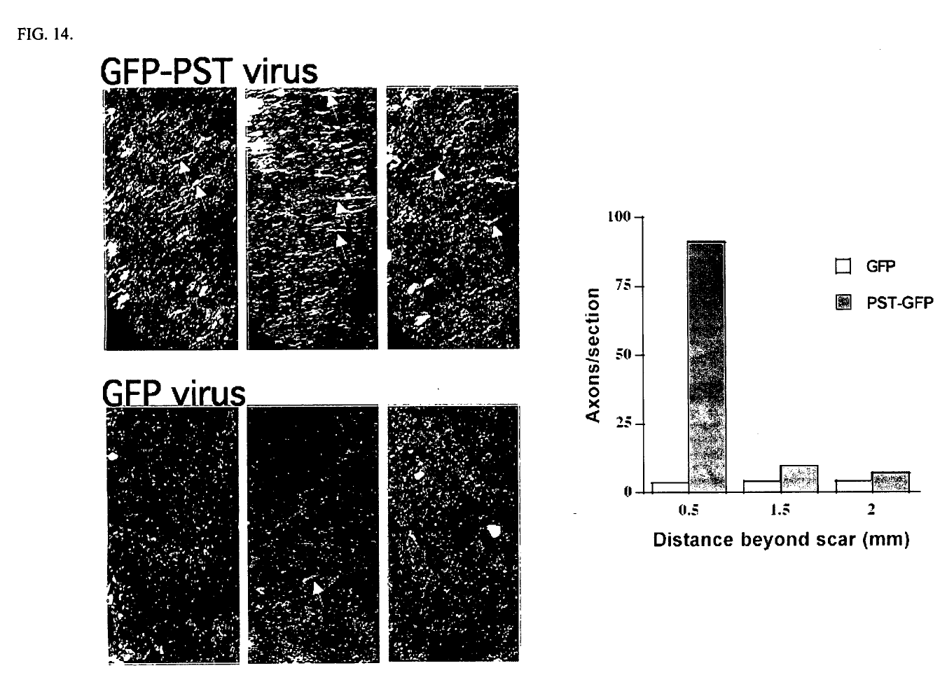

To test the functionality of the construct, COS-7 cells (ATCC, Manassas, Va.) that do not express either PSA or NCAM were transfected (Lipofectamine™ 2000 reagent, Invitrogen, CA) with the pCS-CG plasmid encoding GFP (control) or pCS-CG-PST encoding a GFP-PST fusion protein (FIG. 1A). An additional plasmid encoding human NCAM-140 (GenBank Acc. No. AA364465), a substrate for PST, was also introduced in the cells. The COS-7 cells were cultured under 5% CO₂ in 85% DMEM, 4,500 mg/L D-glucose, 1% L-glutamine, 0.1 mM non-essential amino acids, 10% FBS, and 1% Penicillin/Streptomycin. Western blotting of cell lysates was used to verify expression of the enzyme and its product, as previously described (Canger and Rutishauser, 2004). The PSA-specific 5A5 monoclonal antibody was also used for immunocytochemical assessment of PSA expression in cell cultures. Digestion by the PSA-specific endoneuraminidase N (Endo N, produced and purified from a PK1E phage) (20 U/ml) was used to confirm the presence of the carbohydrate.

C. Production and Testing of Viral Particles

The production of the replication incompetent HIV (ALSV-A) viral vector was achieved by transfecting 3 plasmids into 293T cells (ATCC) using calcium chloride (Lewis et al., 2001): the transfer vector pCS-CG (GFP) or pCS-CG-(GFP-PST), the plasmid pCB6 WTA encoding the ALSV-A receptor and vesicular stomatitis virus VSVG (Lewis et al., 2001), and the plasmid pCMV AR8.2 encoding HIV accessory proteins. The three original plasmids were kindly provided by Drs. Brian Lewis and Harold Varmus at Memorial Sloan-Kettering Cancer Center. Seventy-two hours after transfection of 293T cells, the culture medium containing viral particles was concentrated by centrifugation (50,000×g for 2 hours 30 min). The supernatant was removed and the pellet resuspended in 300 μl TNE-buffer (50 mM Tris-HCl pH 8.0, 130 mM NaCl, 11 mM EDTA). The virus was then aliquoted and stored at −80° C.

To assess the titer of the virus preparation, DF-1 cells (ATCC, VA) were infected with 0.1 μl, 1 μl or 10 μl ( 1/10,000, 1/1,000 or 1/100 respectively) of the GFP-PST or the GFP virus, in the presence of polybrene (8 μg/ml) (Sigma, Mo.). The cells were incubated for 48 hours then observed under a fluorescence microscope for GFP expression. The virus titer was determined by counting the GFP expressing (infected) cells and was approximately 1-2×10⁷ IU/ml for both GFP-PST and GFP viruses. To test the efficiency of the viral constructs in brain slices of adult TVA animals, brains were dissected, transferred in MEM-HG/10% FCS with Penicillin/Streptomycin, 350 μm vibratome slices were cut and incubated in the same medium plus 8 μg/ml polybrene in the presence of the GFP or GFP-PST virus at dilutions of 1/100, 1/1,000, 1/100,000. After 6 days culture at 37° C. under 5% CO₂, the slices were fixed in 4% paraformaldehyde at 4° C. for 2 hours, washed, then processed for immunostaining.

D. Virus Injection

Adult 6 month transgenic GFAP-TVA-mice (gift of Dr. Eric Holland, Memorial Sloan Kettering Cancer Center, New York, N.Y.) were anesthetized with Ketamine (100 mg/kg) and Xylazine (10 mg/kg). The cranium was trephinated 1.7 mm anterior from the bregma and 0.6 mm lateral from the midline using a high-speed micro-drill (0.9 mm) (F.S.T, CA). An atraumatic Hamilton syringe (needle 3PK, 22MM/PT.3; Hamilton Co., Reno Nev.) was introduced 3.8 mm deep (from the calvarium) through the cortex and CC to the edge of the SVZ. This minimal lesion did not induce glial cell division (data not shown). 1 μl of the viral preparation (encoding GFP [n=8] or GFP-PST [n=8]) were injected by the same syringe at 0.2 μl/min during slow retraction of the needle towards the cortical surface. Handling of the virus and animals was in accordance with institutional guidelines.

E. Immunocytochemistry

Under deep anesthesia, the animals were perfused (4% paraformaldehyde in 0.1M phosphate buffer, pH 7.4), and the brains or spinal cords fixed in 4% paraformaldehyde overnight. Sagittal vibratome slices (50 μm) were incubated with primary antibody overnight at 4° C. After washing with PBS and incubation 2 hours at room temperature with appropriate secondary antibodies, the slices were mounted in mowiol (Calbiochem, CA). Primary antibodies used were mouse monoclonal anti-PSA (5A5, produced in the inventors' laboratory, 1:2,000); mouse monoclonal anti-GFP (1:500; Roche, Germany); mouse monoclonal anti-nestin (0.5 μg/ml; Chemicon International, Temecula, Calif.); mouse monoclonal anti-GFAP antibody (5 μg/ml; Chemicon International, CA); mouse monoclonal anti-NeuN (1:150; Chemicon International, CA, Cat # MAB377); rabbit polyclonal anti-NSE (neuron specific enolase (GenBank Acc. No. M22565), 1:100, Chemicon International, CA, Cat. #, AB951); rabbit polyclonal anti-TH (tyrosine hydroxylase (GenBank Acc. No. X53503 or X53577)), 1:150; Pel-Freez, A R, Cat # P40101-0; rabbit polyclonal anti-MAG (myelin-associated glycoprotein, 1:25; Zymed, CA); mouse monoclonal anti-CNPase (2′,3′-cyclic nucleotide 3′-phosphodiesterase; GenBank Acc. No. NM009923) 1:150 (Sternberg monoclonals, Lutherville, Md., Cat # SMI 91); mouse monoclonal anti-NF-M (neurofilament-M (145K), 1:200; Chemicon International, CA), rabbit polyclonal anti-GAD 65 (glutamate decarboxylase, 1:200, Chemicon International, CA), goat polyclonal anti-ChAT (choline acetyltransferase, 1:100; Chemicon International, CA) and mouse monoclonal anti-04 (1:150; Chemicon International, CA). Secondary antibodies were FITC-, Cy3- and Cy5-conjugated (Jackson ImmunoResearch Laboratories, Inc., West Grove, Pa.). Immunostaining with secondary antibodies alone was used as a negative control. The stained tissue was imaged with a confocal microscope (LSM 510; Zeiss). An ANOVA statistical analysis of cell numbers in the CC was carried out using StatView™. In certain instances colocalization of markers in the same cell was confirmed by examining the staining in both the z and y axis using the orthogonal feature of the LSM510 software with a series of confocal images taken at 0.5-0.6 μm intervals through regions of interest.

F. Mitotic Labeling of Tissue

Animals were injected intraperitoneally with bromodeoxyuridine (BrdU; 25 mg/ml in water; Sigma Chemical Co., St. Louis, Mo.) at 100 mg/kg body weight directly after operation and at postoperative days 7, 14, 21, 28 and 2 hours before sacrifice at day 30. Vibratome slices (50 μm) were pre-treated with 1N HCl, 0.4% triton-X 0.5% BSA (in 0.1M phosphate buffer pH 7.4) at 37° C. for 1 hour and then immunostained with a rat FITC-conjugated anti-BrdU antibody (1:20; Serotec, United Kingdom).

G. Induction of Apoptosis in the Mouse Cortex

After injecting the virus in the adult GFAP-TVA animals as described above, 0.5 μl ibotenic acid (Sigma-Aldrich; 0.1 mg/ml in water) was injected into the cortex of the animals. The stereotactic parameters lateral and anterior from bregma were the same as used for the viral injections (1.7 mm anterior and 0.7 mm lateral from bregma). The deepness of the injection however was 1.5-2 mm from the calvarium. The 0.5 μl ibotenic acid was injected during a 5 min interval.

H. Counting of Cells Migrating into the Corpus Callosum and Cortex

The intermediate filament protein nestin is a predominant marker for stem and progenitor cells in the mammalian CNS. BrdU and nestin double stained cells were observed under the confocal microscope and counted in the lesioned CC and the cortex. Ten consecutive sections (40 μm thick, area of 1 mm²) were counted for every animal taking into account the epicenter of the lesion as well as the adjacent 200 μm. The mean of the counted cells/slice was used for ANOVA (analysis of variance) statistical analysis.

I. Induction of a Corticospinal Lesion

4-5 month old male mice were placed under deep pentobarbital anesthesia. The right corticospinal tract (CST) of each mouse was carefully and completely lesioned using the tip of a fine needle (26G1/2) around the level of the 10^{th} dorsal vertebra. Simultaneously, 1 μl of a high titer viral solution (approximately 1-2×10⁷ IU/ml; either PST-GFP, or GFP virus as control) was slowly injected at the lesion site using an atraumatic Hamilton syringe (FIG. 11). Five weeks later, the corticospinal axons were anterogradely labeled by stereotaxic injection of fluoro-ruby (tetramethylrhodamine-labeled 10,000 Da dextran, lysine fixable; 10 mg/ml; Molecular Probes, Eugene, Oreg.) in the sensori-motor cortex (Coumans et al., 2001). Fluoro-ruby stains regenerating corticospinal axons. In the images shown here the red fluorescence is visualized in a confocal channel that shows the stained axons as blue. Animals were sacrificed at 6 weeks by transcardial perfusion with a 4% paraformaldehyde solution in 0.1 M phosphate buffer, pH 7.4.

### Example 1

### PSA Expression in COS-7 Cells after Transfection with the GFP-PST Plasmid

The GFP-PST fusion protein was inserted into the pCS-CG plasmid (FIG. 1A), and tested by co-transfection into COS-7 cells along with the NCAM plasmid. The Western blot shows a GFP-positive band at the expected molecular weight of 27 kDa in an extract of COS-7 cells transfected with GFP (control; FIG. 1B, left blot, left lane). Cells transfected with GFP-PST fusion protein yielded a band at 69 kDa (FIG. 1B, left blot, right lane), which is the expected molecular weight for the fusion protein. On the right (FIG. 1B, right blot), a Western blot specific for PSA shows a band at molecular weights over 150 kDa in NCAM/GFP-PST transfected COS-7 cells (center lane). These cells normally lack NCAM-PSA and are unable to express PSA after co-transfection with NCAM DNA only (control, left lane). The PSA band disappeared when the lysate was treated for 30 min with the PSA-specific endoneuraminidase N(PST+endo N, right lane).

Polysialylation of NCAM as also evidenced by immunostaining of the cells (FIG. 1C, center right). In FIG. 1C, all the GFP panels show green fluorescence (left panels), whereas only the central row, stained for GFP-PST (right panels), shows red fluorescence. Treatment of the transfected cells with endo N completely removed PSA (FIG. 1C, bottom row, right panel), and polysialylation did not occur when the GFP-PST construct was replaced by a control GFP plasmid (FIG. 1C, top row, right panel). Thus FIG. 1C shows that the NCAM/GFP transfected cells did not express PSA, whereas the NCAM/GFP-PST transfected cultures expressed high levels of PSA, and PSA was completely removed by endo N.

### Example 2

### Viral-Infection and PST Expression in Astrocytes of Adult GFAP-TVA transgenic mice

GFAP-TVA transgenic animals express the TVA receptor exclusively in astrocytes, which allows selective infection of these cells by HIV (ALSV-A) lentiviral vectors. Accordingly, when brain slices from these animals were incubated with lentivirus carrying either the GFP-PST or the GFP construct, GFP expression (green) was restricted to only to astrocytes (red; GFAP positive) (FIG. 1D). After 6 days in culture, PSA appeared on the surface of the GFP-PST-infected astrocytes (data not shown).

After the in vitro studies, virus encoding GFP or GFP-PST was injected in vivo in the transgenic mice along a needle track extending through the cortex and corpus callosum (CC) to the SVZ (FIG. 2A). Immunohistological examination of the injected regions after 30 days revealed that the virus had selectively infected GFAP-expressing astrocytes (FIG. 2B), which have been reported to be found at lesioned areas (Fawcett and Asher, 1999). PSA was absent both in uninfected regions and in regions infected only with the GFP virus (FIG. 2B). This experiment shows that the GFP-injected controls and the uninjected samples had no PSA expression on the surface of GFAP-positive cells, while high levels of PSA expression occurred following transfection with GFP-PST. When the GFP-PST virus was used, the ensuing PSA expression co-localized with that of GFAP (FIG. 2 C). The left image shows GFAP staining in blue, and the center image shows PSA staining in red. The merged image (right) shows essentially complete superposition of GFAP (left) and PSA (center) expression as brighter shapes, indicating that GFAP-positive cells were the only cells expressing PSA 30 days after injection.

### Example 3

### PSA Expression Enhances SVZ Progenitor Migration into the Corpus Callosum

To determine whether ectopic PSA expression would influence the migration of SVZ cells, experiments similar to those in Example 2 were carried out using the GFAP-TVA transgenic mice. The progenitor cells were pre-labeled by repetitive BrdU injections to identify cells undergoing DNA replication and cell proliferation, and were visualized by co-staining for BrdU and nestin (FIG. 3). Injection of the control GFP virus alone resulted in deviation of a small but significant number of SVZ progenitors into the CC as compared to an uninjected animal. (The induced expression of PSA in the control studies involves a needle track lesion, which can increase the presence of SVZ precursors in white matter (Goings et al., 2004)). Injection of the PST construct, on the other hand, increased the migration of progenitor cells into the CC by more than 4 times (FIGS. 3A and 3B; p<0.001). The injections did not induce glial cell division, since GFAP-labeled cells were not BrdU-positive (data not shown). Precursors that had migrated to white matter were able to develop nestin-positive processes (FIG. 3 A, lower right image) as has also been observed for SVZ cells that have just arrived at their normal destination in the olfactory bulb (Petridis et al., 2004). The expression of PSA by astrocytes extended beyond the CC into the cortex (FIG. 4A), indicating expression of similar levels of PSA in the CC and cerebral cortex. BrdU-labeled progenitor cells migrated into the CC (FIG. 4B), confirming the results shown in FIG. 3, but were not able to migrate along the PSA-positive zone in the cortex under the conditions of these experiments (FIG. 4B).

### Example 4

### SVZ Progenitors Differentiate within the Corpus Callosum

The fate of migrating SVZ progenitor cells was examined in GFAP-TVA transgenic mice. Normal migration of SVZ progenitor cells along the rostral migratory stream (RMS) towards the olfactory bulb is shown in FIG. 5 A. The stained cells are immunopositive for BrdU (green, left; many stained spots within the rostral migratory stream (RMS)) and nestin (red, center; many stained spots within the RMS) and are small and round, localized primarily in the injection track. The merged images show that these staining centers superimpose (FIG. 5A, right; bright or light features within the RMS).

Unlike the progenitors in the SVZ rostral migratory stream, the nestin/BrdU-positive cells in the CC produced long nestin-positive processes (FIGS. 3 A and 5B, center (red stained processes throughout CC region)). The merged image (FIG. 5B, right) shows that many of the nestin-positive processes are also BrdU-positive (bright, light spots in the CC).

To further characterize the differentiation state of the BrdU-labeled cells in the CC, immunostaining for NeuN (a marker for early postmitotic neurons; Mullen et al., 1992; Magavi et al., 2000), 04 (a marker for immature oligodendrocytes; Zhang, 2001), NSE or TH (markers for mature olfactory interneurons; Eriksson et al., 1998; Betarbet et al., 1996), and CNPase or MAG (markers for mature oligodendrocytes; Zhang, 2001) was carried out. The percent of BrdU stained cells in the CC costaining for other markers is shown in FIG. 6C. About 40% of BrdU-labeled cells (stained green in FIGS. 6A and 6B) also expressed O4 (stained red in FIG. 6A, left image; superimposed stains show bright, light spots), approximately two-thirds expressed CNPase (stained red in FIG. 6A, center image; a large red area surrounds a central bright, light spot), and one-third BrdU-positive cells also expressed NeuN (stained red in FIG. 6B, left image (large arrow); bright, light spot shows BrdU-positive nucleus), but none displayed NSE or TH (FIG. 6A-C). Cells also stained for MAG (FIG. 6A, right image; large red spots surrounding bright, light spots showing BrdU-positive nuclei). The arrows in FIG. 6A, center and right images, indicate processes of CNPase- and MAG-positive oligodendrocytes. MAG staining was used to confirm the identity of CNPase-positive cells. A 3D (stereoscopic) reconstruction of the BrdU co-labeling with CNPase or MAG corroborates the superposition of the BrdU stain with the CNPase and MAG stains (not shown). No BrdU-positive cells expressed TH (tyrosine hydroxylase), which is normally expressed by SVZ cells when they become interneurons in the olfactory bulb. Thus, most progenitors had become or were destined to become mature oligodendrocytes, whereas the remainder had adopted a neuronal fate but were unable or delayed in their ability to become mature neurons (see FIG. 6D for a schematic diagram of this model). (There is no MAG immunoreactivity of the environment immediately surrounding the injection path since myelin was destroyed by this procedure. Regions of the CC away from the injected area were highly immunoreactive for MAG.)

The results described in Examples 1-4 indicate that induction of PSA on astrocytes near the SVZ-CC border promotes migration of SVZ precursors into the CC, where they primarily differentiate into oligodendrocytes. The effects described in the Examples reflect increased expression of PSA on astrocytes in the CC environment. Without wishing to be bound by theory, it is believed that the induced PSA may serve to enhance migration by reducing adhesions or contact-dependent repulsive signals produced by non-neuronal cells in this environment.

An increased number of precursor cells is not useful unless they can differentiate into cell types appropriate to their environment. Others have shown that, upon arrival at the olfactory bulb, SVZ cells grow nestin-positive neurites (Petridis et al., 2004) and then differentiate into TH-positive interneurons (Betarbet et al., 1996). In the present Examples the SVZ cells that were induced to migrate into the CC by PSA expression also grew nestin-positive processes, but then mostly followed a glial maturation pathway leading to mature oligodendrocytes, in contrast to the results of Betarbet et al. (1996). This capacity of induced precursor cells to migrate and differentiate into oligodendrocytes in the corpus callosum has not been observed previously. (In other work, differentiation of precursor cells according to their environment has been observed in hippocampal ischemic lesions, where the precursors become pyramidal cells (Nakatomi et al, 2002), and after experimentally-induced demyelination or encephalomyelitis, in which they become oligodendrocytes (Picard-Riera et al., 2002; Decker et al., 2002)).

In Examples 5-7 below, a model system for brain lesions characterized by focal neural destruction, such as caused by stroke or trauma, is developed and characterized. The lesions are induced by the injection of ibotenic acid.

### Example 5

### Induction of a Cortical Lesion

The neurotoxic compound ibotenic acid (alpha-amino-3-oxo-4-isoxazoline-5-acetic acid; Sigma-Aldrich) was injected into the cerebral cortex of transgenic GFAP-TVA mice in order to produce a focal lesion. The lesion included apoptotic cells, as shown by annexin-V immunostaining that remains detectable 3 weeks after injection (FIG. 7A), whereas injection of PBS induced no apoptosis (FIG. 7B). This chemical lesion also induced local GFAP expression (data not shown) in control animals injected with PBS only, as well as a small amount of PSA (FIG. 7B). When the neurotoxin-induced lesion was combined with viral PST infection so that the needle passed through the lesion on the way to the SVZ, high levels of PSA were obtained on astrocytes all along the track (FIG. 7B), whereas only low levels of PSA were detected following ibotenic acid treatment in conjunction with control virus.

### Example 6

### Effect of Cortical Lesion on Migration of Progenitors

Transgenic mice were treated with ibotenic acid as in Example 5, and injected with PST virus or control virus. Combination of the chemical lesion with the PST-virus injection protocol resulted in migration of markedly increased numbers of progenitor cells that are both BrdU- and nestin-positive over the entire path from the SVZ to the lesion (FIG. 8A, right column). There was also what appeared to be a robust tangential dispersion of these cells away from the needle track and into the cortex, roughly corresponding to the region affected by chemical lesion and exhibiting PSA expression (FIG. 7A). This shows that PSA over-expression promotes progenitor migration into the chemically induced cortical lesion. By contrast, the chemical lesion combined with the control injection produced a much smaller number of BrdU-positive cells, and uninjected cortices had a minimal number of progenitors (FIG. 8A, left column). Quantification of the number of BrdU/nestin-positive cells in the chemical cortical lesion shows that infection of the needle track astrocytes with the PST virus resulted in a large increase in progenitor migration into the cortical lesion (FIG. 8B); the chemical lesion alone (control) produces a modest number of progenitors as compared to uninjected cortices.

### Example 7

### Differentiation of Migrating Progenitor Cells

The brains of transgenic mice that had been lesioned with ibotenic acid and injected with viral PST were probed immunohistochemically for markers of differentiation. The results are shown in FIG. 9A, which includes, in the panels labeled ii. and iii., overlays of staining by BrdU (for cell proliferation) in green and neurofilament-M (NF-M, a marker for neurons) in red, with superposed intensity showing as light or bright spots. It is seen in panels iii. that several long red-staining (NF-M) processes (pointed out by several arrowheads) emanate from the bright centers (pointed out by full arrows) showing coincident staining by both NF-M and BrdU. By contrast to animals whose brains were only injected with viral PST, but not with ibotenic acid (see Example 4), in the cortex of animals treated both with PST virus and ibotenic acid, 74% of the BrdU-positive cells were also NF-M positive (FIG. 9C), including lateral to the injection route as well as at the epicenter of the injection (evident in a 3D stereoscopic reconstruction, not shown). BrdU-positive cells in the chemically lesioned cortex were positive for NF-M, or ChAT (FIG. 9B). Most BrdU positive cells were TH negative. Some TH positive axons were present in the lesioned area, however they do not belong to the BrdU-positive cells. 23% of cells were CNPase-positive and only 3.5% were GFAP-positive (FIG. 9C). Therefore, most of these progenitors were in the process of differentiating into mature neurons. Normally SVZ progenitors that migrate to the OB become TH-positive interneurons (Betarbet et al., 1996). However, in the cortical environment the BrdU-positive cells stained for CHAT (22) % or GAD (8%) but only 1.5% were TH-positive (FIGS. 9B and 9C; these are enzyme markers for the synthesis of neurotransmitters). The chemically induced cortical lesion did not cause a significant proliferation of GFAP-positive astroglial cells (1.5%; other data not shown).

The results in Examples 6 and 7 indicate that induced expression of PSA on astrocytes in an experimental path bridging the SVZ and a CNS lesion in the cortex can enhance migration of progenitor cells to the region of the lesion in a manner that has not been previously observed. Furthermore, the results in Examples 1-7 show that precursors induced to undergo migration by exposure to PSA decoration of NCAM are able to acquire distinct fates according to their ultimate environment. The precursor cells that were induced to appear in the CC by PSA expression grew nestin-positive processes, but then predominantly followed a glial maturation pathway leading to mature oligodendrocytes. By contrast, a primarily neuronal fate was adopted by progenitors that appeared in the gray matter in response to both induced PSA expression and the chemical lesion. These results are summarized in the schematic diagram of FIG. 10. Panel A diagrams a limited amount of PSA induction and precursor recruitment that is produced by a lesion alone. Panel B, Left: illustrates that expression of PSA (red) alone on the surface of astrocytes causes a robust but short-range recruitment into the corpus callosum with subsequent differentiation into oligodendrocytes. The Right image illustrates that addition of a chemical lesion such as that induced by ibotenic acid (including apoptosis) results in enhanced migration of precursors far into the cortex; these cells are capable of assuming a neuronal fate. In these experiments, it is believed that the BrdU-positive precursors originate in the SVZ because in the absence of the chemical lesion the BrdU labeling is confined to the white matter just adjacent to the SVZ, whereas substantial numbers of progenitors in the gray matter were only obtained when PSA-expression was induced in mice that had been subjected to the chemical lesion protocol.

In sum, Examples 1-7 above demonstrate that induction of PSA on glial cells on a route connecting a lesion with the SVZ has the two major requirements of an approach to endogenous progenitor cell therapy: it increases the number of progenitors available to the injury site and provides them in a state amenable to differentiate according to local environmental cues.

Examples 8-10 below illustrate that artificial over-expression of PSA in astrocytes of the corticospinal tract (CST) promotes axonal regrowth through an inhibitory glial scar resulting from a corticospinal lesion.

### Example 8

### Stimulated Production of PSA in Astrocytes in Mice with a Spinal Cord Lesion

Transgenic mice expressing the TVA receptor exclusively in glial cells were lesioned and infected by an HIV pseudotyped ALSV-A virus carrying a coding sequence for PST (see Example 2). In this model, PSA-NCAM was selectively expressed in scar astrocytes in the region studied in these Examples.

A corticospinal lesion was created as follows. 4-5 month old male mice were placed under deep pentobarbital anesthesia. The right corticospinal tract (CST) of each mouse was carefully and completely lesioned using the tip of a fine needle (26G1/2) around the level of the 10^{th} dorsal vertebra. Simultaneously, 1 K11 of a high titer viral solution (approximately 1-2×10⁷ IU/ml; either PST-GFP virus or GFP virus control) was slowly injected at the lesion site using an atraumatic Hamilton syringe (FIG. 11). Five weeks later, the corticospinal axons were anterogradely labeled by stereotaxic injection of fluoro-ruby (tetramethylrhodamine-labeled 10,000 Da dextran, lysine fixable; 10 mg/ml; Molecular Probes, Eugene, Oreg.), which stains regenerating corticospinal axons, in the sensori-motor cortex (Coumans et al., 2001). Animals were sacrificed at 6 weeks by transcardial perfusion with a 4% paraformaldehyde solution in 0.1 M phosphate buffer, pH 7.4.

It is believed that the surgical lesion leads to activation of astrocytes, which in turn produce an elevated induction of GFAP promoter; this thereby increases expression of the TVA receptor and the susceptibility of infection of astrocytes by the virus. FIG. 12, top panels, show images from control samples injected with virus harboring only GFP. It is seen that the green GFP-expressing astrocytes are found at the lesion site (upper left image, green spots appear throughout the image, concentrated in the lower half; upper right image, green spots are concentrated at the lower center of the image). The bottom panels show glial scars from PST-GFP-infected animals, which exhibit high levels of PSA (red; areas of highest red color are outlined by white borders). This Example shows that high expression of PSA chains was observed in the glial scar of spinal cords treated with PST-GFP virus as compared to GFP controls. PSA was expressed on infected astrocytes and their processes.

### Example 9

### Axons Grow Through the PSA-Expressing Scar

Transgenic mice were treated as described in Example 8 to induce a corticospinal lesion. FIG. 13, top panel shows an image from a control section (i.e., infected with GFP-only virus) of fluoro-ruby-labeled corticospinal axons, showing axons (blue, toward the right of the image) at the entrance of the glial scar (green, toward the lower left of the image). The axons showed a high number of dystrophic growth cone-bearing sprouts (see the white-line bordered images, and arrow). No axonal sprouts are growing inside the glial scar (green). The arrow below the image indicates the rostro-distal axis (direction of axon growth). This observation, serving as a control in this Example, is in agreement with previous work (Cajal, 1928; Tom et al., 2004).

In FIG. 13, the bottom set of four images shows PSA production in PST-GFP-infected glial scars. Note the presence of extended axons (blue, upper left and both lower panels, several of which are outlined by white lines) growing in contact with PSA-expressing cells (red, upper right and both lower panels) inside the scar (in the upper right image selected regions staining intense red are outlined by white lines). The frequency of dystrophic cones decreased in PSA-expressing scars of PST-GFP-infected animals; instead, normal growth cones were encountered growing in contact with PSA expressing surfaces of astrocytes inside the lesion. The arrows in the PST-infected images point to axon profiles of growing neurites.

### Example 10

### Quantitation of Axons Projecting Beyond a Corticospinal Lesion

Transgenic mice infected with HIV(ALSV-A) vectors harboring GFP-PST or GFP only were subjected to corticospinal trauma, as in Example 8. FIG. 14 shows that there are a high number of axon profiles in cross section distal to the lesion in PST-GFP treated mice (top set of three images; see, for example, the arrows) as compared to GFP-treated controls (bottom set of three images). Thus, a higher number of axons were encountered in the CST area distal to the lesion in PST-GFP samples, whereas fewer axonal profiles could be found in control sections. Axons in successive slices distal to the lesion were enumerated. Quantitation of axons able to project beyond the distal border of the scar revealed that about 25 times more axons (P<0.001) were able to grow through the scar in animals treated with PST-GFP vector, as compared to GFP controls (FIG. 13, right panel). It is concluded that expression of PST at the site of lesion in the PST-GFP transfected mice leads to overexpression of PSA-modified NCAM, which allows a large number of axons to grow through and exit the distal border of the scar. Importantly, the PSA-expressing scar does not inhibit axon regeneration.

Analysis of further transverse sections at successive distal levels showed that once the growing axons have exited the PSA-positive scar, their growth was retarded. As shown in FIG. 14, right panel, a significantly high number of axons were able to penetrate across the scar and grow distally up to 0.5 mm in PST-GFP samples. However, in the experiments reported here, counts of regenerating axons dropped to control levels at 1.5 and 2 mm. This dropoff at longer distances is not surprising, since the viral treatment was localized at the lesion site and PSA was observed to be over-expressed mainly in the gliotic scar (FIG. 12). It is believed that the myelin inhibitory barrier (Filbin, 2003) that lies distal to the scar remains unaffected under the conditions of the experiments reported in this Example, and may cause regeneration to abort.

Overall, the regrowth of corticospinal axons through the gliotic scar occurs at levels 25 times greater than in control lesioned animals. This regrowth of axons across neuronal scar tissue has not been reported before.

In summary, the results reported in Examples 8-10 clearly show that PSA can be over-expressed in adult astrocytes at CNS lesion sites. This PSA over-expression renders astrocytes able to support the extension of regenerating CST axons through the inhibitory scar environment. This new approach, together with other methods to overcome myelin inhibition in areas distal to the lesion, could be a useful tool in treatment of CNS lesions. It is believed that this is the first procedure to accomplish such regrowth through the scar tissue.

Examples 1-10 provide results indicating that a CNS disease or pathology may be treated by bridging the distance between the CNS lesion and the origin of CNS precursors, in regions such as the subventricular zone or the dentate gyrus in the hippocampus, via a path where PSA or PSA-NCAM is elevated. Such PSA or PSA-NCAM elevation can be accomplished in many ways, including those disclosed in the present invention.

### Example 11

### Preparation of Broad-Spectrum Lentivirus Harboring PST

To infect cells with PST in non-TVA (i.e., wild type) animals, a vector comprising GFP-polysialyltransferese ST8Sia IV (GFP-PST) inserted into a replication incompetent HIV (vesicular stomatitis virus G (VSVG) vector (Miller et al., 1996)) has been employed. The TOPO Cloning procedure (Invitrogen Corp., Carlsbad, Calif.) was carried out according to the manufacturer. cDNA encoding GFP-PST was cloned under the CMV promoter into the pLenti6/V5-TOPO expression vector. The engineered construct was packaged into the ViraPower Lentiviral Expression System (Invitrogen Corp.) to produce a high-titer viral particle solution. This virus is designed to infect both dividing and non-dividing mammalian cells both in vivo and in vitro using a replication-incompetent lentivirus. This vector provides high-level gene expression in a wide variety of cell types, including the astrocytes and neurons involved in the present invention, as well as keratinocytes and fibroblasts. This GFP-PST vector has been demonstrated to express high levels of PSA on NCAM in non-TVA cell lines.

### Example 12

### Polysialylation of Cell Surface Components

Cell surface glycolipids are polysialylated in vitro by contacting mammalian cells exhibiting cell surface glycosphingolipids such as GD3, GT1a, GQ1b, GT1b, GD2, GD1b, GD1a, and GM1 with any of a variety of PST's. For example, the PST of Escherichia coli K1 has been used (Cho et al., 1994). In addition, the gene sequence encoding this protein (identified in GenBank Acc. No. X60598) may be cloned and expressed to provide the protein in situ, in in vitro cell cultures, or in vivo. Polysialylation of cell surface components is envisioned in the present invention to provide a means for stimulating migration of neural progenitor cells to a site in the CNS at which they may exert a beneficial therapeutic effect.

### Example 13

### Preparation of Specific PSA-Antibody Conjugates Targeting Cell Surface

Components

PSA is conjugated with a specific immunoglobulin, such as IgG, IgM, or an F(ab)₂ fragment by the sodium meta-periodate method. The antibody or antibody fragment has been raised by immunization with a cell surface protein of interest or a fragment thereof that is immunogenic, and specifically binds that cell surface protein. Such a protein may be an intercellular adhesion molecule, a neural cell adhesion molecule, and integrin, a cadherin, or a particular cell surface receptor.

The periodate method of conjugation involves the reaction of sodium meta-periodate with the vicinal diol groups present in sialic acid (the monosaccharide building block of PSA) resulting in the formation of a vicinal dialdehyde. This dialdehyde reacts with free amino groups present on protein the immunoglobulin at alkaline pH of 9.0-9.5 and yields a Schiff base, which in turn is stabilized by reduction using sodium cyanoborohydride. An example of a conjugation protocol follows.

1. To 5 mg PSA (Sigma Chemical Co.) add 1 ml of 0.1 M Sodium acetate buffer pH 5.5 and dissolve well.

2. To the above solution add 62.5 μl (2.5 mg; 11.675 μmole) of sodium meta-periodate in 0.1 M sodium acetate buffer pH 5.5 and stir at room temperature for 1 h 30 min.

3. At the end of incubation period add 3 μl (2.899 mg) of ethylene glycol (4× molar excess) as a quencher and allow to stand at R.T for 15 min with stirring.

4. Remove excess NaIO₄ by passing through Sephadex G1 column equilibrated with acetate buffer. Collect 1 ml fractions, test the fractions for presence of PSA, and combine fractions that showed positive for PSA.

5. To the reaction mixture add 5 mg IgG dissolved in carbonate buffer; adjust the pH of the reaction between 9 and 9.5. Stir at 37° C. for 15-30 min.

6. Add 2 mg (30.22 μmole) of sodium cyanoborohydride and continue the incubation at 37° C. or room temperature for over night.

7. Remove the unreacted IgG and PSA by using Sephacryl S-300 (or Bio Gel A 0.5 m) column with phosphate buffer pH 7.2-7.4 or suitable buffer. Test the eluted fractions for the simultaneous presence of PSA and protein. Combine fractions positive for both PSA and protein as the product conjugate.

### Example 14

### Analysis of a PSA-Anti-NCAM Conjugate

A PSA-anti-NCAM IgG was prepared using the procedure of Example 13. It was characterized on SDS-PAGE immunoblots with anti-IgG and anti-PSA antibodies and found to be a polydisperse IgG+/PSA+ band of high molecular size, typical of PSA-protein conjugates.

The PSA-anti-NCAM IgG was also shown to bind to the surface of NCAM+ cells (such as NCAM-transfected COS cells) by showing intense labeling of the conjugate-treated cells with anti-PSA antibody.

All patents, patent application publications, and patent applications identified herein are incorporated by reference in their entireties, as if appearing herein verbatim. All technical publications identified herein are also incorporated by reference.

Although the present invention may have been disclosed and illustrated herein by reference to exemplary embodiments thereof, all equivalent embodiments, including alterations, additions and omissions, are encompassed within the spirit and scope of the invention including the claims.

### REFERENCES

Angata K, Fukuda M (2003) Polysialyltransferases: major players in polysialic acid synthesis on the neural cell adhesion molecule. Biochimie 85:195-206.
Arvidsson A, Collin T, Kirik D, Kokaia Z, Lindvall 0 (2002) Neuronal replacement from endogenous precursors in the adult brain after stroke. Nat Med. 8(9):963-970.
Barnett and Riddell. 2004. J Anat. 204(1):57-67
Batcholor and Howells. 2003. J Clin Neurosci.; 10(5):523-34.
Bates P, Young J A, Varmus H E (1993) A receptor for subgroup A Rous sarcoma virus is related to the low density lipoprotein receptor. Cell 74:1043-1051.
Bayer S A, Yackel J W, Puri P S (1982) Neurons in the rat dentate gyrus granular layer substantially increase during juvenile and adult life. Science 216(4548):890-892.
Behar T N, Dugich-Djordjevic M M, Li Y X, Ma W, Somogyi R, Wen X, Brown E, Scott C, McKay R D, Barker J L (1997) Neurotrophins stimulate chemotaxis of embryonic cortical neurons. Eur J Neurosci. 9(12):2561-2570.
Benangiano V, Virgintino D, Flace P, Girolamo F, Errede M, Roncali L, Ambrosi G (2003). Choline acetyltransferase-containing neurons in the human parietal neocortex. Eur J Histochem. 47(3):253-256.
Benraiss A, Chmielnicki E, Lerner K, Roh D, Goldman S A (2001) Adenoviral brain-derived neurotrophic factor induces both neostriatal and olfactory neuronal recruitment from endogenous progenitor cells in the adult forebrain. J Neurosci. 21(17):6718-6731.
Betarbet R, Zigova T, Bakay R A, Luskin M B (1996) Dopaminergic and GABAergic interneurons of the olfactory bulb are derived from the neonatal subventricular zone. Int J De
Bradbury E J, Moon L D, Popat R J, King V R, Bennett G S, Patel P N, Fawcett J W, McMahon S B. 2002. Nature. 416(6881):636-40;
Cajal S G, 1928. Degeneration and regeneration of the nervous system Vols 1 & 2. Oxford University Press, London.
Calza L, Giardino L, Pozza M, Bettelli C, Micera A, Aloe L (1998) Proliferation and phenotype regulation in the subventricular zone during experimental allergic encephalomyelitis: In vivo evidence of a role for nerve growth factor. Proc. Natl. Acad. Sci. 95:3209-3214.
Canger A K, Rutishauser U (2004) Alteration of neural tissue structure by expression of polysialic acid induced by viral delivery of PST polysialyltransferase. Glycobiology 14(1):83-93.
Chalmers G R, Peterson D A, Gage F H. 1996. J Comp Neurol. 371(2):287-99.
Cho, J W, and Troy, F A, Proc Natl Acad Sci USA. 1994 91(24): 11427-11431.
Coumans J V, Lin T T, Dai H N, MacArthur L, McAtee M, Nash C, Bregman B S. 2001. J Neurosci. 21(23):9334-44.
Cremer H, Lange R, Christoph A, Plomann M, Vopper G, Roes J, Brown R, Baldwin S, Kraemer P, Scheff S et al (1994) Inactivation of the NCAM gene in mice results in size reduction of the olfactory bulb and deficits in spatial learning. Nature 367:455-459.
Daston M M, Bastmeyer M, Rutishauser U, O'Leary D D. 1996. J Neurosci. 16(17):5488-97.
Decker L, Durbec P, Rougon G, Evercooren A B (2002) Loss of polysialic residues accelerates CNS neural precursor differentiation in pathological conditions. Mol Cell Neurosci. 19(2):225-238.
Doetsch F, and Alvarez-Buyfla A. 1996. Network of tangential pathways for neuronal migration in adult mammalian brain. Proc. Natl. Acad. Sci. USA. 93: 14895-14900.
Dusart I, Morel M P, Wehrle R, Sotelo C. 1999. J Comp Neurol. 408(3):399-418.
El Maarouf, A., and Rutishauser, U. 2003. J. Comp. Neurol. 460, 203-211.
Eriksson P S, Perfilieva E, Bjork-Eriksson T, Alborn A M, Nordborg C, Peterson D A, Gage F H (1998) Neurogenesis in the adult human hippocampus. Nat Med. 4(11): 1207.
Fallon J, Reid S, Kinyamu R, Opole I, Opole R, Baratta J, Korc M, Endo T L, Duong A, Nguyen G, Karkehabadhi M, Twardzik D, Loughlin S (2000). In vivo induction of massive proliferation, directed migration, and differentiation of neural cells in the adult mammalian brain. Proc. Natl. Acad. Sci. 97(26): 14686-14691.
Fawcett J W and Asher R A (1999) The glail scar and central nervous system repair. Brain Research Bulletin 49(6):377-391.
Filbin M T. 2003. Nat Rev Neurosci. 4(9):703-13.
Franceschini I A, Barnett S C. 1996. Dev Biol. 173(1):327-43.
Franceschini I, Vitry S, Padilla F, Casanova P, Nam Tham T, Fukuda M, Rougon G, Durbec P, Dubois-Dalcq M (2004) Migrating and myelinating potential of neural precursors engineered to overexpress PSA-NCAM. Mol Cell Neurosci. 27(2):151-162.
Fujimoto, I., Bruses, J. L. and Rutishauser, U. 2001. J. Biol. Chem. 276, 31745-31751.
Goings G E, Sahni V, Szele F G (2004) Migration patterns of subventricular zone cells in adult mice change after cerebral cortex injury. Brain Research 996:213-226.
Grimpe B, and Silver J. 2004. J Neurosci. 24(6):1393-7.
Holland E C, Varmus H E (1998) Basic fibroblast growth factor induces cell migration and proliferation after glia-specific gene transfer in mice. Proc Natl Acad Sci USA 95(3):1218-1223.
Hoyk Z, Parducz A and Theodosis D T. 2001. European Journal of Neuroscience, 13, 649-56.
Hu H, Tomasiewicz H, Magnuson T, Rutishauser U (1996) The role of polysialic acid in migration of olfactory bulb interneuron precursors in the subventricular zone. Neuron 16(4):735-43.
Kaplan M S, Hinds J W (1977) Neurogenesis in the adult rat: electron microscopic analysis of light radioautographs. Science 197(4308):1092-1094.
Kelly S, Bliss T M, Shah A K, Sun G H, Ma M, Foo W C, Masel J, Yenari M A, Weissman I L, Uchida N, Palmer T, Steinberg G K (2004). Transplanted human fetal neural stem cells survive, migrate, and differentiate in ischemic rat cerebral cortex. Proc Natl Acad Sci USA 101(32): 11839-11844.
Kendall B E (1993) Inborn errors and demyelination: MRI and the diagnosis of white matter disease. J Inherit Metab Dis. 16(4):771-786.
Laywell E D, Dorries U, Bartsch U, Faissner A, Schachner M, Steindler D A. 1992. Proc Natl Acad Sci USA. 89(7):2634-8.
Lemons M L, Howland D R, Anderson D K. 1999. Exp Neurol. 160(1):51-65.
Lewis B C, Chinnasamy N, Morgan R A, Varmus H E (2001) Development of an avian leukosis-sarcoma virus subgroup A pseudotyped lentiviral vector. J Virol. 75(19):9339-9344.
Li Y, Field P M, Raisman G. 1997. Science. 277(5334):2000-2.
Magavi S S, Leavitt B R, Macklis J D (2000) Induction of neurogenesis in the neocortex of adult mice. Nature 405:951-955.
Magavi S S, Macklis J D (2002). Induction of neuronal type-specific neurogenesis in the cerebral cortex of adult mice: manipulation of neural precursors in situ. Developmental Brain Research 134:57-76.
Miller, A. D., Proc. Natl. Acad. Sci. USA 93, 111407-11413 (1996).
Miyoshi, H., U. Blomer, M. Takahashi, F. H. Gage, and I. M. Verma. 1998. Development of a self-inactivating lentivirus vector. J. Virol. 72:8150-8157.
Moon L D, Asher R A, Rhodes K E, Fawcett J W. 2001. Nat Neurosci. 4(5):465-6.
Morel M P, Dusart I, Sotelo C (2002) Sprouting of adult Purkinje cell axons in lesioned mouse cerebellum: “non-permissive” versus “permissive” environment. J Neurocytol. 31(8-9):633-647.
Mullen R J, Buck C R, Smith A M (1992) NeuN, a neuronal specific nuclear protein in vertebrates. Development 116(1):201-211.
Nakatomi H, Kuriu T, Okabe S, Yamamoto S, Hatano O, Kawahara N, Tamura A, Kirino T, Nakafuku M (2002) Regeneration of hippocampal pyramidal neurons after ischemic brain injury by recruitment of endogenous neural progenitors. Cell 110(4):429-441.
Nakayama J, Fukuda M (1995). A human polysialyltransferase directs in vitro synthesis of polysialic acid. J. Biol. Chem. 271(4):1829-1832.
Nakayama J, Angata K, Ong E, Katsuyama T, Fukuda M. 1998. Pathol Int. 48(9):665-77.
Nothias F, Vernier P, von Boxberg Y, Mirman S and Vincent J D. 1997. European Journal Neuroscience, 9, 1553-65.
Ohmiya M, Shudai T, Nitta A, Nomoto H, Furukawa Y, Furukawa S (2002) Brain-derived neuritrophic factor alters cell migration of particular progenitors in the developing mouse cerebral cortex. Neurosci Lett. 317(1):21-24.
Oda Y, Nakanishi 1 (2000). The distribution of cholinergic neurons in the human central nervous system. Histol Histopathol. 15(3):825-834.
Ono K, Tomasiewicz H, Magnuson T, Rutishauser U (1994) N-CAM mutation inhibits tangential neuronal migration and is phenocopied by enzymatic removal of polysialic acid. Neuron 13(3):595-609.
Oumesmar B N, Vignais L, Duhamel-Clerin E, Avellana-Adalid V, Rougon G, Baron-Van Evercoren A (1995). Expression of the highly polysialylated neural cell adhesion molecule during postnatal myelination and following chemically induced demyelination of the adult mouse spinal cord.
Petridis A K, El-Maarouf A, Rutishauser U (2004) Polysialic acid regulates cell contact-dependent neuronal differentiation of progenitor cells in the subventricular zone. Dev Dyn. 230(4):675-684.
Picard-Riera N, Decker L, Delarasse C, Goude K, Nait-Oumesmar B, Liblau R, Pham-Dinh D, Evercooren A B (2002) Experimental autoimmune encephalomyelitis mobilizes neural progenitors from the subventricular zone to undergo oligodendrogenesis in adult mice. Proc Natl Acad Sci USA 99(20):13211-13216.
Rudge J S, Silver J. 1990. J Neurosci. 10(11):3594-603.
Rutishauser, U. and Landmesser, L. 1996. Trends Neurosci. 19, 422-427.
Rutishauser, U. 1996. Curr. Opin. Cell Biol. 8, 679-684.
Seki, T. and Rutishauser, U. 1998. J. Neurosci. 18, 3757-3766.
Solberg Y, White E L, Keller A (1988). Types and distribution of glutamic acid decarboxylase (GAD)-immunoreactive neurons in mouse motor cortex. Brain Res. 459(1): 168-172.
Theodosis D T, Bonhomme R, Vitiello S, Rougon G and Poulain D A. 1999. Journal of Neuroscience, 19, 10228-36.
Tom V J, Steinmetz M P, Miller J H, Doller C M, Silver J. 2004. J. Neurosci. 24(29):6531-9.
Wang, C., Rougon, G. and Kiss, J. Z. 1994. J. Neurosci. 14, 4446-4457.
Yamamoto, N., Inui, K., Matsuyama, Y., Harada, A., Hanamura, K., Murakami, F., Ruthazer, E. S., Rutishauser, U. & Seki, T. 2000. J. Neurosci. 20, 9145-9151.
Yang, P., Yin, X. and Rutishauser, U. 1992. J. Cell. Biol. 116, 1487-1496.
Yin X, Watanabe M, Rutishauser U. 1995. Development. 121(10):3439-46.
Yoshida, K., Rutishauser, U., Crandall, J. E. and Schwarting, G. A. 1999. J. Neurosci. 19, 794-801.
Young J A, Bates P, Varmus H E (1993) Isolation of a chicken gene that confers susceptibility to infection by subgroup A avian leukosis and sarcoma viruses. J. Virol. 67: 1811-1816.
Zhang S C (2001) Defining glial cells during CNS development. Nat Rev Neurosci. 2(11):840-843.
Zigova T, Pencea V, Wiegand S J, Luskin M B (1998) Intraventricular administration of BDNF increases the number of newly generated neurons in the adult olfactory bulb. Mol Cell Neurosci. 11(4):234-245.

1. A method of promoting migration of a first neural cell or portion thereof from an originating region toward a second region, comprising contacting a second neural cell with a composition that is effective to decorate the second neural cell with a hydrated polymer.

2. The method described in claim 1 wherein the second neural cell is located substantially within a zone comprising the second region.

3. The method described in claim 1 wherein the second neural cell is located substantially within a zone comprising the originating region.

4. The method described in claim 1 wherein the second neural cell is located substantially within a zone comprising at least the originating region, the second region and a volume extending between them.

5. The method described in claim 1 wherein the second neural cell comprises a glial cell.

6. The method described in claim 1 wherein the second neural cell comprises a neuronal cell.

7. The method described in claim 1 wherein the hydrated polymer comprises a hydrogel.

8. The method described in claim 1 wherein the hydrated polymer comprises a polysaccharide or a derivative thereof.

9. The method described in claim 1 wherein the hydrated polymer comprises a polymer chosen from the group consisting of a polysialic acid, a hyaluronic acid, a polylactosamine, or a derivative of any of them.

10. The method described in claim 1 wherein the composition comprises a polynucleotide encoding a polypeptide having activity that synthesizes a hydrated polymer bound to a moiety on an external surface of the second neural cell, or a complement thereof.

11. The method described in claim 10 wherein the composition comprises a plasmid, a vector, or a virus particle that comprises the polynucleotide, wherein the plasmid, vector, or virus particle enters the second neural cell and expresses the polynucleotide therein.

12. The method described in claim 10 wherein the polypeptide comprises a polysialyltransferase, an ortholog thereof, a variant thereof, or a fragment of any of them.

13. The method described in claim 11 wherein the activity synthesizes a hydrated polymer bound to a moiety on the external surface of the second neural cell.

14. The method described in claim 13 wherein the moiety is a protein.

15. The method described in claim 13 wherein the moiety is a neural cell adhesion molecule.

16. The method described in claim 1 wherein the composition comprises a polysialyltransferase, or any of an ortholog thereof, a variant thereof, a fragment of any of the foregoing, a peptidomimetic, or a mimetic compound, that exhibits polysialyltransferase activity.

17. The method described in claim 16 wherein the activity synthesizes a hydrated polymer bound to a moiety on the external surface of the second neural cell.

18. The method described in claim 17 wherein the moiety is a protein.

19. The method described in claim 17 wherein the moiety is a neural cell adhesion molecule.

20. The method described in claim 1 wherein the composition comprises a conjugate comprising the hydrated polymer bound to a targeting means wherein the targeting means binds to a moiety of the external surface of the second neural cell.

21. The method described in claim 20 wherein the targeting means comprises a lectin or an antibody.

22. The method described in claim 1 wherein the first neural cell is a neural progenitor cell.

23. The method described in claim 22 wherein the originating region comprises at least a portion of a subventricular zone or a dentate gyrus in the hippocampus.

24. The method described in claim 22 wherein the second region comprises at least a portion of corpus callosum.

25. The method described in claim 22 wherein the second region comprises at least a portion of cortex.

26. The method described in claim 22 wherein the second region comprises a lesion.

27. The method described in claim 1 wherein the portion of a first neural cell comprises an outgrowth of a neuronal cell.

28. The method described in claim 27 wherein the second region comprises a lesion of the central nervous system.

29. The method described in claim 28 wherein the originating region comprises at least a portion of a neurite tract from which outgrowth occurs.

30. A method of treating or ameliorating a neurological pathology in a subject, wherein the method comprises contacting a second neural cell with a composition that is effective to decorate a second neural cell with a hydrated polymer, wherein the second neural cell is located substantially within a zone of interest comprising at least one of an originating region, a second region, and a space between them.

31. The method described in claim 30 wherein the hydrated polymer comprises a polysaccharide or a derivative thereof.

32. The method described in claim 30 wherein the composition comprises a polynucleotide encoding a polypeptide having activity that synthesizes a hydrated polymer bound to an external surface of the second neural cell, or a complement thereof.

33. The method described in claim 30 wherein the composition comprises a polysialyltransferase, or any of an ortholog thereof, a variant thereof, a fragment of any of the foregoing, a peptidomimetic, or a mimetic compound, that exhibits polysialyltransferase activity.

34. The method described in claim 30 wherein the composition comprises a conjugate comprising the hydrated polymer bound to a targeting means wherein the targeting means binds to a moiety of the external surface of the second neural cell.

35. The method described in claim 30 wherein the originating region contains a first neural cell or portion thereof.

36. The method described in claim 35 wherein the first neural cell comprises a neural progenitor cell.

37. The method described in claim 35 wherein the portion of the first neural cell comprises an outgrowth of a neuronal cell.

38. The method described in claim 30 wherein the pathology is a CNS lesion.

39. The method described in claim 38 wherein the lesion is the result of a stroke or a traumatic injury.

40. The method described in claim 30 wherein the pathology is a CNS neurite tract lesion.

41. A conjugate comprising a hydrated polymer bound to a targeting means wherein the targeting means binds to a moiety of the external surface of a neural cell.

42. The conjugate described in claim 41 wherein the targeting means comprises a lectin or an antibody.

43. The conjugate described in claim 41 wherein the hydrated polymer comprises a polysaccharide or a derivative thereof.

44. A lentiviral vector comprising a polynucleotide encoding a polypeptide having activity that synthesizes a hydrated polymer bound to a moiety wherein the moiety displays the hydrated polymer on an external surface of a mammalian cell, or a complement thereof, wherein the vector enters the cell and expresses the polynucleotide therein.

45. The vector described in claim 44 wherein the polypeptide comprises a polysialyltransferase, an ortholog thereof, a variant thereof, or a fragment of any of them.

46. The vector described in claim 44 wherein the mammalian cell is a neural cell.

47. The vector described in claim 44 wherein the moiety is a protein.

48. The vector described in claim 44 wherein the moiety is a neural cell adhesion molecule.

49-50. (canceled)
